(19) United States
(12) Patent Application Publication
(10) Pub. No.: US 2026/0242871 A1
(43) Pub. Date: Aug. 20, 2026

(54) PLASMA CELL-FREE RNA SIGNATURES OF TUBERCULOSIS

(71) Applicant: CORNELL UNIVERSITY, Ithaca, NY (US)

(72) Inventors: Iwijn DE VLAMINCK, Ithaca, NY (US); Adrienne ZAGIEBOYLO, Ithaca, NY (US); Conor LOY, Ithaca, NY (US); Daniel EWEIS-LABOLLE, Ithaca, NY (US)

(73) Assignee: CORNELL UNIVERSITY, Ithaca, NY (US)

(21) Appl. No.: 19/134,922

(22) PCT Filed: Dec. 1, 2023
(86) PCT No.: PCT/US2023/082046

§ 371 Date: Jun. 2, 2025

Related U.S. Application Data
(60) Provisional application No. 63/429,733, filed on Dec. 2, 2022.

(51) Int. Cl. C12Q 1/6883; G16B 40/20; G16H 50/20

(52) U.S. Cl. CPC C12Q 1/6883; G16B 40/20; G16H 50/20; C12Q 2600/118; C12Q 2600/158

(57) ABSTRACT

The current disclosure current disclosure is directed to methods of diagnosing, triaging, and treating tuberculosis. Another aspect of the current disclosure is directed to methods of establishing a panel of biomarkers that correlates with tuberculosis disease status.

### CROSS REFERENCE TO RELATED APPLICATION

This application claims the benefit of priority from U.S. Provisional Application No. 63/429,733, filed Dec. 2, 2022, the entire contents of which are incorporated herein by reference.

### STATEMENT REGARDING FEDERALLY SPONSORED RESEARCH OR DEVELOPMENT

This invention was made with government support under Grant Nos. R01AI146165, R21AI133331, R21AI124237, and R01AI151059 awarded by the National Institutes of Health. The government has certain rights in the invention.

### BACKGROUND

Tuberculosis (TB) remains a leading cause of death from an infectious disease worldwide. This is partly due to a lack of tools to effectively screen and triage individuals with potential TB. The outcome of infection after exposure to Mycobacterium tuberculosis, the causative agent TB, can vary greatly and is determined by a complex interplay between host immunity and bacterial persistence. Current tests for TB are not sensitive to the full spectrum of disease states, are unable to determine if an infection has been cleared, cannot distinguish between latent, incipient, and subclinical disease, or predict progression to active TB. The lack of informative and minimally invasive tests presents a major challenge in the management and control of TB.

Transcriptomic signatures, or changes in host-cell gene expression, can provide valuable insight into the host response to TB. Whole blood transcriptomic signatures have been extensively investigated as biomarkers for TB but have failed to meet the optimal target product profiles (TPPs) recommended by the World Health Organization (WHO) for a non-sputum based triage or diagnostic test. Additionally, the most promising whole blood transcriptomic signature, RISK 11, performed poorly outside of South Africa, and was not useful for targeting short-course TB preventive therapy.

Plasma cell-free RNA (cfRNA) is an analyte that can provide information beyond what can be achieved with conventional whole blood RNA (wbRNA) signatures. cfRNA is released by cells in both the blood compartment and from vascularized solid tissues, and thus contains information about systemic immune dynamics and immune-tissue interactions. In contrast, wbRNA largely reflects RNA expression of intact leukocytes. wbRNA is predominantly derived from live cells, whereas cfRNA is primarily released by dying cells. As such, cfRNA may provide insights into pathways of cell death and mechanisms of cellular injury that would not be available with wbRNA profiling.

### SUMMARY

The current disclosure is directed to methods of diagnosing, triaging, and treating tuberculosis as well as methods of establishing a panel of biomarkers that correlates with tuberculosis disease status.

An aspect of the current disclosure is directed to a method comprising:
a) obtaining cell-free RNA from a biological sample of a subject, wherein the biological sample is a plasma sample or a serum sample;
b) determining a profile of a panel of biomarkers in the cell-free RNA; and
c) determining a tuberculosis disease status in the subject based on the profile of the panel of biomarkers.

In some embodiments, the step of determining a profile of a panel of biomarkers comprises performing nucleotide sequencing of the cell-free RNA. In some embodiments, the step of determining a profile of a panel of biomarkers comprises measuring the levels of RNAs corresponding to the biomarkers in the panel. In some embodiments, the panel of biomarkers comprises one or more genes selected from the group consisting of GBP5, CCND1, JADE1, ALB, NFIA, KAT6B, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, CST3, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, GOSR1. In some embodiments, the panel of biomarkers comprises one or more genes selected from macrophage markers, neutrophil markers, interferon genes, or antimicrobial genes. In some embodiments, the macrophage markers comprise of one or more genes selected from the group consisting of MARCO, SOCS3, FCGR1A, MPO, and C1QB. In some embodiments, the neutrophil markers comprise of one or more genes selected from the group consisting of ELANE, FCGR3A, S100A8, S100A9, and ERG. In some embodiments, the interferon genes comprise of one or more genes selected from the group consisting of IFI27L1, IFIT2, IFIT3, IFITM3, and IRF1. In some embodiments, the antimicrobial genes comprise of one or more genes selected from the group consisting of AZU1, CTSG, DEFA4, STAT1, GBP1, GBP2, GBP4, GBP5, and GBP6. In some embodiments, the panel of biomarkers comprises GBP5. In some embodiments, the panel of biomarkers comprises GBP5 and one or more additional genes. In some embodiments, the panel of biomarkers comprises GBP5 and 2-8 additional genes. In some embodiments, the panel of biomarkers comprises GBP5 and one or more genes selected from LBH, KAT6B, FKBP5, CCND1, Clorf115, and FCER1G; or DYSF, SMARCD3, VAMP5, FCGR3B, GPI, MPO, CREB5, and GBP2. In some embodiments, the panel of biomarkers comprises one or more genes selected from the group consisting of GBP5, LBH, KAT6B, FKBP5, CCND1, Clorf115, and FCER1G; or GBP5, DYSF, SMARCD3, VAMP5, FCGR3B, GPI, MPO, CREB5, and GBP2. In some embodiments, determining a tuberculosis disease status in the subject based on the profile of the panel of biomarkers comprises comparing the profile of the panel of biomarkers in the cell free RNA from the biological sample to a reference profile of the panel of biomarkers. In some embodiments, determining a tuberculosis disease status in the subject based on the profile of the panel of biomarkers comprises comparing the levels of RNAs corresponding to the biomarkers in the panel to levels of RNAs corresponding to the biomarkers in the reference profile. In some embodiments, a tuberculosis disease status comprises a tuberculosis free status, a latent tuberculosis status, an active tuberculosis status, or a tuberculosis progressing status. In some embodiments, the biological sample is a plasma sample. In some embodiments, the biological sample is a serum sample. In some embodiments, the subject is a mammal. In some embodiments, the subject is a human. In some embodiments, the profile of the panel of biomarkers comprises subject-specific gene expression of the biomarkers in the panel. In some embodiments, the profile of the panel of biomarkers comprises a score determined based on the levels of RNAs corresponding to the biomarkers in the panel using a trained classification model, and wherein the score correlates with disease status. In some embodiments, the cell-free RNA is substantially free of contaminant RNA.

In some embodiments, the tuberculosis disease status determined is used to triage the subject. In some embodiments, the tuberculosis disease status determined is used in determining treatment for the subject. In some embodiments, the tuberculosis disease status determined is used to monitor treatment of the subject. In some embodiments, the tuberculosis disease status determined is used to monitor efficacy of a treatment of the subject. In some embodiments, test performance of the trained classification model is measured through one or more of accuracy, sensitivity, specificity, and/or area under the receiver operating characteristic curve (ROC-AUC).

In some embodiments, the tuberculosis disease status is determined at a greater than 80%, 85%, 90%, 91%, 92%, 93%, 94%, or 95% accuracy. In some embodiments, the tuberculosis disease status is determined at a greater than 80%, 85%, 90%, 91%, 92%, 93%, 94%, or 95% sensitivity. In some embodiments, the tuberculosis disease status is determined at a greater than 70%, 75%, 80%, 85%, 90%, 91%, 92%, 93%, 94%, or 95% specificity. In some embodiments, the trained classification model comprises one or more models selected from the group consisting of generalized linear models with Ridge and LASSO feature selection (GLMNETRIDGE and GLMNETLASSO), support vector machines with linear and radial basis function kernel (SVMLin and SVMRAD), random forest (RF), random forest ExtraTrees (EXTRATREES), neural networks (NNET), linear discriminant analysis (LDA), nearest shrunken centroids (PAM), C5.0 (C5), k-nearest neighbors (KNN), naive bayes (NB), CART (RPART), generalized linear model (GLM), and greedy forward search algorithm (GFS).

In some embodiments, the method further comprises step d) treating the subject based on the tuberculosis disease status. In some embodiments, step d) treating the subject based on the tuberculosis disease comprises treating the subject with an antibiotic.

An aspect of the disclosure is directed to a method for establishing a panel of biomarkers that correlates with tuberculosis disease status, comprising
a) obtaining cell-free RNA from a biological sample of each of a group of subjects, wherein the biological sample is a plasma sample or a serum sample, and wherein the group of subjects comprise subjects positive for tuberculosis and subjects negative for tuberculosis;
b) detecting levels of cell-free RNAs of a plurality of genes in the subjects;
c) dividing the subjects into a training set and a testing set, wherein each set comprises subjects positive for tuberculosis and subjects negative for tuberculosis;
d) based on levels of cell-free RNAs of a plurality of genes in the training set, selecting a panel of genes, wherein the selecting comprises:
   selecting differentially abundant genes based on a differential analysis; and
   training a classification model using the levels of cell-free RNAs of the selected genes; thereby determining a classification score threshold for optimal classifier performance in an ROC (Receiver Operating Characteristics) analysis;
e) testing the trained classification model by applying the trained model and the classification score threshold to the cell-free RNA levels of the testing set, and determining the panel of genes as the panel of biomarkers that correlates with tuberculosis disease status wherein the trained classification model provides differentiation in the testing between subjects positive for tuberculosis and subjects negative for tuberculosis with at least 85% sensitivity and at least 65% specificity. In some embodiments, the differentially abundant genes are selected by removing genes of low count and genes having an adjusted p-value greater than 0.05. In some embodiments, the classification model comprises one or more models selected from the group consisting of generalized linear models with Ridge and LASSO feature selection (GLMNETRIDGE and GLMNETLASSO), support vector machines with linear and radial basis function kernel (SVMLin and SVMRAD), random forest (RF), random forest ExtraTrees (EXTRATREES), neural networks (NNET), linear discriminant analysis (LDA), nearest shrunken centroids (PAM), C5.0 (C5), k-nearest neighbors (KNN), naive bayes (NB), CART (RPART), generalized linear model (GLM), and greedy forward search algorithm (GFS). In some embodiments, the group of subjects include at least 100 subjects positive for TB, and at least 100 subjects negative for TB. In some embodiments, the panel of biomarkers comprises at least 1 gene. In some embodiments, the trained classification model provides differentiation in the testing between subjects positive for tuberculosis and subjects negative for tuberculosis with at least 80% sensitivity. In some embodiments, the trained classification model provides differentiation in the testing between subjects positive for tuberculosis and subjects negative for tuberculosis with at least 60% specificity. In some embodiments, the trained classification model provides differentiation in the testing between subjects positive for tuberculosis and subjects negative for tuberculosis with at least 90% sensitivity and at least 70% specificity. In some embodiments, step c) further comprises dividing the subjects into a training set, a validation set, and a testing set, wherein each set comprises subjects positive for tuberculosis and subjects negative for tuberculosis. In some embodiments, step (e) further comprises testing the trained classification model by applying the trained model and the classification score to the cell-free RNA levels of the validation set and the testing set, and determining the panel of genes as the panel of biomarkers that correlates with tuberculosis disease status if the trained classification model provides differentiation in the testing between subjects positive for tuberculosis and subjects negative for tuberculosis with at least 85% sensitivity and at least 65% specificity. In some embodiments, when the trained classification model does not provide differentiation in the testing between subjects positive for tuberculosis and subjects negative for tuberculosis with at least 85% sensitivity and at least 65% specificity, the method further comprises one or a combination of the steps:
f) selecting a different modeling technique;
g) adjusting the hyperparameters for training the model;
h) changing the initial filtering steps and how Differential abundance analysis is performed; and/or
i) adjusting the classifier score threshold based on the training set.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1G. Plasma cell-free RNA profiling. FIG. 1A) Geographic distribution of samples included in this study, which originated from three clinical studies. FIG. 1B) Differences in cfRNA cell-types-of-origin between the three cohorts. FIG. 1C) Scaled counts per million values of significantly differentially abundant genes (DESeq2, Benjamini-Hochberg adjusted p-value<0.05. Samples and genes are clustered based on correlation. FIG. 1D) Top 25 differential pathways between microbiologically-confirmed TB diagnoses ranked by significance. Z-score indicated in corresponding bar; direction relative to TB negative samples. FIG. 1E) Flowchart of the method used to train, test, and validate machine learning classification algorithms. FIG. 1F) Area under the receiver operating characteristic curve (ROC-AUC) metrics for training and test sets for each machine learning classification algorithm.

FIGS. 2A-2F. Performance of the 7-gene signature identified in plasma cfRNA. FIG. 2A) Area under the receiver operating characteristic curve (ROC-AUC) as a function of the gene added in each iteration of a greedy forward search model performed on the training dataset. FIG. 2B) Train, test, and validation performance of the greedy forward search algorithm in distinguishing microbiologically confirmed TB. FIG. 2C) Violin plot of classifier scores for training, test, and validation sets using the greedy forward search algorithm. FIG. 2D) Abundance of GBP5 by cohort and Xpert Ultra Semi-quantitative Result. Outliers are indicated with arrows and values. FIG. 2E) Correlation of the 7-gene TB score with the Xpert Ultra Semi-quantitative Results (dotted line: classification score threshold=0.528; bars indicate 95% confidence interval). FIG. 2F) Correlation of the 7-gene TB score with three chest X-ray scores. Color indicates disease status (pink=TB positive; blue=TB negative).

FIGS. 3A-3D. Comparison of the 7-gene plasma signature with whole blood signatures of TB from literature. FIG. 3A) Overlap between top-performing whole blood signatures (blue) and the 9-gene signature (pink). Top 30 overlapping genes are shown. FIG. 3B) Performance comparison of whole blood signatures (blue) with the 7-gene signature (pink). Optimal triage thresholds are marked in blue, minimal triage thresholds are marked in green. FIG. 3C) Performance of GBP5 mRNA abundance in distinguishing active TB. FIG. 3D) Performance comparison of whole blood protein GBP5, whole blood RNA GBP5, and plasma cfRNA GBP5 in distinguishing active TB.

FIG. 4. Differences in the cell-types of origin of plasma cfRNA and whole blood RNA. Solid organ fraction measured in plasma cfRNA [END TB (blue), R2D2 (magenta), and TB2 Plasma (green)] and a matched whole blood cohort [TB2 Whole Blood (orange)]. ****adjusted p-value<0.0001.

### DETAILED DESCRIPTION

The current disclosure is directed to methods of diagnosing, triaging, and treating tuberculosis (TB). Using RNA sequencing and machine learning, the disclosed methods enabled a signature that can accurately distinguish individuals with microbiologically diagnosed TB. The diagnostic accuracy, sensitivity, and specificity of the signature compare favorably to the performance of whole blood signatures of previously known methods. The methods of the disclosure utilize cfRNA as a host-specific blood-borne biomarker for TB. The use of plasma cfRNA as a bioanalyte to triage, monitor, and diagnose TB as disclosed herein is important. The signatures derived from plasma cfRNA of a subject may be more robust than those derived from whole blood RNA (wbRNA), which have shown poor performance in independent validation studies. This may be due to the sensitivity of whole blood signatures to differences in subject cohorts and sample processing. The disclosed methods using plasma cfRNA signature produced robust against the sensitivity of whole blood signatures, making it a potentially more reliable source for TB biomarker discovery. Also, plasma cfRNA is stable, can be assayed using a small amount of plasma, and can provide insight into host cellular injury and death.

Although claimed subject matter will be described in terms of certain examples, other examples, including examples that do not provide all the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

In the description that follows, certain conventions will be followed as regards to the usage of terminology. Generally, terms used herein are intended to be interpreted consistently with the meaning of those terms as they are known to those of skill in the art. In practicing the present disclosure, many conventional techniques in molecular biology, microbiology, cell biology, biochemistry, and immunology are used, which are within the skill of the art. These techniques are described in greater detail in, for example, Molecular Cloning: a Laboratory Manual 4th edition, J. F. Sambrook and D. W. Russell, ed. Cold Spring Harbor Laboratory Press 2012; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the disclosure.

An aspect of the current disclosure is directed to a method comprising:
a) obtaining cell-free RNA (cfRNA) from a biological sample of a subject, wherein the biological sample is a plasma sample or a serum sample;
b) determining a profile of a panel of biomarkers in the cell-free RNA; and
c) determining a tuberculosis disease status in the subject based on the profile of the panel of biomarkers.

The term “biological sample” includes body samples from an animal, including biological fluids such as serum, plasma, vitreous fluid, lymph fluid, synovial fluid, follicular fluid, seminal fluid, amniotic fluid, milk, whole blood, urine, cerebro-spinal fluid, saliva, sputum, tears, perspiration, mucus, and tissue culture medium, as well as tissue extracts such as homogenized tissue, and cellular extracts. In some embodiments, the biological sample is a serum, plasma or urine sample. In some embodiments, the biological sample is a plasma sample. In some embodiments, the biological sample is a serum sample.

The term “subject” refers to mammals. Non-limiting examples of mammals include but are not limited to human, horse, camel, dog, cat, pig, cow, goat, and sheep. In some embodiments, the mammal is human.

The terms “cell-free RNA” or “cfRNA” refer to cfRNA released by cells in both the blood compartment and from vascularized solid tissues. cfRNA contains information about systemic immune dynamics and immune-tissue interactions. cfRNA is primarily released by dying cells; therefore, cfRNA may provide insights into pathways of cell death and mechanisms of cellular injury. cfRNA is also released into the blood by way of active secretion. cells release cfRNA to the subject's bodily fluid, and thus may increase the quantity of the specific cfRNA in the subject's biological sample as compared to a healthy individual. cfRNA may include any types of RNA that are circulating in the bodily fluid of a person without being enclosed in a cell body or a nucleus.

Extraction of cfRNA is known in the art. Briefly, in some embodiments, plasma samples are received on dry ice and stored at −80° C. until processed. Prior to cfRNA extraction, plasma samples are thawed at room temperature and centrifuged. cfRNA is extracted from plasma (300-1,000 μl) using the Norgen Plasma/Serum Circulating and Exosomal RNA Purification Mini Kit (Norgen, 51000). Extracted RNA is treated with DNase Turbo Buffer (Invitrogen, AM2238), DNase Turbo (Invitrogen, AM2238), and Baseline Zero DNase (Lucigen-Epicenter, DB0715K), then concentrated into aliquots using the Zymo RNA Clean and Concentrated Kit (Zymo, R1015).

In some embodiments, sequencing libraries are prepared from concentrated RNA using the Takara SMARTer® Stranded Total RNA-Seq Kit v3—Pico Input Mammalian (Takara, 634485) and barcoded using the SMARTer® RNA Unique Dual Index Kit (Takara, 634451). In some embodiments, library concentration was quantified using the Qubit™ 3.0 Fluorometer (Invitrogen, Q33216) with the dsDNA HS Assay Kit (Invitrogen, Q32854). In some embodiments, libraries are quality-controlled using the Agilent Fragment Analyzer 5200 (Agilent, M5310AA) with the HS NGS Fragment Kit (Agilent, DNF-474-0500) and pooled to equal concentrations. Each pool was sequenced using both the Illumina NextSeq 500/550 platform (paired-end, 150 bp) and the Illumina NextSeq 2000 platform (paired-end, 100 bp).

A “gene panel” or “panel” refers to a defined selection of genes that are enriched or sequenced. A panel will include relevant pathogen associated genes and likely variants of selected genes. In a next generation sequencing (NGS) panel test, only clinically important genes are examined to obtain genomic data in a timely and cost-effective manner. In some embodiments, the panel comprises a collection of signature genes. As used herein, the term “signature gene” refers to a gene whose expression is correlated, either positively or negatively, with disease extent or outcome or with another predictor of disease extent or outcome.

As used herein, the term “profile” generally refers to gene expression profile. A gene expression profile can be understood to mean a pattern of abundance of expression of genes. In some embodiments, a gene expression profile is a measurement of the expression of multiple genes at once. In some embodiments, gene expression is measured by cfRNA in a subject, e.g., cfRNA in a serum or plasma sample, in which case, the profile is that of a subject's cfRNA. Gene expression profiles can be characteristic or unique to a status of a subject, e.g., a healthy status or a disease status (such as cancer or infectious disease) and can therefore be used to distinguish between different status. In some embodiments, the profile is a profile of genes identified in this disclosure to be associated with TB, which are also referred to herein as TB signature genes. It is to be understood that the profile of TB signature genes in this disclosure have not all been associated with TB previously. TB signature genes of this disclosure were identified using the methods developed and disclosed herein. TB signature genes exhibit differential expression in subjects having TB relative to subjects without TB, for example. The differential expression refers to the difference in abundance of a signature gene in subjects having TB relative to subjects without TB. In some embodiments, the differential expression of a panel of signature genes can be assigned a value or a classification score which is determined based on the methodology described herein, wherein the score correlates the status of TB (also referred to as a TB score herein). Thus, in some embodiments, a profile of genes is a classification score. In some embodiments, a gene expression score (GEX) can be statistically derived from the expression levels of a set of signature genes and used to diagnose a condition or to predict clinical course. In some embodiments, the expression levels of signature genes may be used to predict progression of TB without relying on a GEX. A “signature nucleic acid” is a nucleic acid comprising or corresponding to, in case of cDNA, the complete or partial sequence of a RNA transcript encoded by a signature gene, or the complement of such complete or partial sequence. A signature protein is encoded by or corresponding to a signature gene of the disclosure.

As used herein, the term “reference profile” refers to the profile of genes (e.g., the same genes identified in this disclosure to be associated with TB) in a subject not inflicted with TB.

In some embodiments, machine learning and model training is performed using R (v4.1.3) with the DESeq2 (v1.34.0), Caret (v6.0.90), and pROC (v1.18.0) packages. In some embodiments, sample metadata and count matrices split 70/30 into a training set and a test set, controlling for disease status, HIV status, and cohort to minimize differences in the training and test datasets. In some embodiments, sample metadata and count matrices are split into a training set, validation set, and test set, controlling for disease status, HIV status, and cohort to minimize differences in the datasets.

In some embodiments, features for model training are selected by filtering and differential abundance analysis. First, differential abundance analysis was performed on the raw training counts using DESeq2. Then feature selection was conducted using the output from DESeq2 on retained genes. In some embodiments, genes are excluded that have a base-mean of less than 100 and a Benjamini-Hochberg adjusted p-value greater than 0.05. In some embodiments, genes are excluded that have a base-mean of less than 50 and a Benjamini-Hochberg adjusted p-value greater than 0.05.

In some embodiments, machine learning algorithms are trained using 5-fold cross validation and grid search hyperparameter tuning. In some embodiments, accuracy, sensitivity, specificity, and/or area under the receiver operating characteristic curve (ROC-AUC) are used to measure test performance. In some embodiments, the classification models used are generalized linear models with Ridge and LASSO feature selection (GLMNETRIDGE and GLMNETLASSO), support vector machines with linear and radial basis function kernel (SVMLin and SVMRAD), random forest (RF), random forest ExtraTrees (EXTRATREES), neural networks (NNET), linear discriminant analysis (LDA), nearest shrunken centroids (PAM), C5.0 (C5), k-nearest neighbors (KNN), naive bayes (NB), CART (RPART), and generalized linear models (GLM).

In some embodiments, an additional model is trained and tested using a greedy forward search algorithm (GFS). Briefly, this algorithm iterates over the list of genes and evaluates each individual gene's discriminatory power by training a generalized linear model (GLM). The discriminatory power is evaluated based on a combined score which is calculated as: (AUC+Sensitivity+Specificity), in which sensitivity and specificity are calculated at the optimal youden threshold. The gene that results in the highest score is added to the model. At each subsequent iteration over the list of candidate genes, the algorithm will attempt to add a gene to the model in this way. The algorithm stops when there is no gene in the list that can increase the score by more than 0.01.

In some embodiments, an additional model was trained and tested using a greedy forward search algorithm. Briefly, the algorithm starts with a single gene that provides the most discriminatory power using a tuberculosis score. The tuberculosis score was calculated as:

$$Tuberculosis score = < {log}_{2} ( CPM + 1 ) {>}_{u p regulated} - < {log}_{2} ( CPM + 1 ) {>}_{d ownregulated} ,$$

where CPM is counts per million obtained using the cpm function (edgeR v3.36.0) on raw counts. At each subsequent step, an additional gene is added, and the tuberculosis score is recalculated. The combination of genes at each step that provides the greatest increase in AUC becomes the basis for the next iteration. The search stops when no gene addition increases the AUC.

In some embodiments, the step of determining a profile of a panel of biomarkers comprises performing nucleotide sequencing of the cfRNA. In some embodiments, the step of determining a profile of a panel of biomarkers comprises measuring the levels of RNAs corresponding to the biomarkers or signature genes in the panel. In some embodiments, the panel of biomarkers comprises one or more signature genes. In some embodiments, the biomarkers are selected from the group consisting of GBP5 (Accession #ENSG00000154451.14), CCND1 (Accession #ENSG00000110092.4), JADE1 (Accession #ENSG00000077684.16), ALB (Accession #ENSG00000163631.17), NFIA (Accession #ENSG00000162599.17), KAT6B (Accession #ENSG00000156650.14), H19 (Accession #ENSG00000130600.19), PURA (Accession #ENSG00000185129.8), FKBP5 (Accession #ENSG00000096060.15), FCGR3B (Accession #ENSG00000162747.12), LPP (Accession #ENSG00000145012.14), LTA4H (Accession #ENSG00000111144.10), S100A8 (Accession #ENSG00000143546.10), S100A9 (Accession #ENSG00000163220.11), TMCC2 (Accession #ENSG00000133069.17), LBH (Accession #ENSG00000213626.13), MEA1 (Accession #ENSG00000124733.5), GBP4 (Accession #ENSG00000162654.9), STAT1 (Accession #ENSG00000115415.20), OPA1 (Accession #ENSG00000198836.11), BCL11B (Accession #ENSG00000127152.18), DUSP3 (Accession #ENSG00000108861.9), GBP1 (Accession #ENSG00000117228.11), SYNE2 (Accession #ENSG00000054654.19), DTX3L (Accession #ENSG00000163840.10), DYSF (Accession #ENSG00000135636.15), EIF1 (Accession #ENSG00000173812.11), GABARAP (Accession #ENSG00000170296.10), IFITM3 (Accession #ENSG00000142089.17), RNU5A-1 (Accession #ENSG00000199568.1), SLC2A1 (Accession #ENSG00000117394.24), CAVIN1 (Accession #ENSG00000177469.13), GPI (Accession #ENSG00000105220.17), IGF2 (Accession #ENSG00000167244.21), KLF6 (Accession #ENSG00000067082.15), SERPINB6 (Accession #ENSG00000124570.21), SRSF9 (Accession #ENSG00000111786.9), CST3 (Accession #ENSG00000101439.9), ERG (Accession #ENSG00000157554.19), GCA (Accession #ENSG00000115271.11), LYZ (Accession #ENSG00000090382.7), MNDA (Accession #ENSG00000163563.8), NDUFA5 (Accession #ENSG00000128609.16), ATP6V1F (Accession #ENSG00000128524.5), LMNB1 (Accession #ENSG00000113368.12), PLAAT4 (Accession #ENSG00000133321.11), APOL6 (Accession #ENSG00000221963.6), ENAH (Accession #ENSG00000154380.17), GBP2 (Accession #ENSG00000162645.13), HMGN2 (Accession #ENSG00000198830.11), HEMGN (Accession #ENSG00000136929.13), ATM (Accession #ENSG00000149311.20), USP12 (Accession #ENSG00000152484.14), BNIP3L (Accession #ENSG00000104765.16), PGD (Accession #ENSG00000142657.21), Clorf115 (Accession #ENSG00000162817.7), MLLT6 (Accession #ENSG00000275023.5), ALDOA (Accession #ENSG00000149925.22), WARS1 (Accession #ENSG00000140105.18), ABLIM1 (Accession #ENSG00000099204.20), MTND2P28 (Accession #ENSG00000225630.1), YOD1 (Accession #ENSG00000180667.11), UBE2J1 (Accession #ENSG00000198833.7), ARPC1B (Accession #ENSG00000130429.15), FAM13B (Accession #ENSG00000031003.10), PLAC8 (Accession #ENSG00000145287.11), VPS13C (Accession #ENSG00000129003.19), FECH(ENSG00000066926.13), GPX1 (Accession #ENSG00000233276.8), SNORD3A (Accession #ENSG00000263934.5), GREM1 (Accession #ENSG00000166923.12), USP4 (Accession #ENSG00000114316.13), YTHDC1 (Accession #ENSG00000083896.13), ZC3H6 (Accession #ENSG00000188177.14), FCER1G (Accession #ENSG00000158869.11), SH3BGRL3 (Accession #ENSG00000142669.15), SNX3 (Accession #ENSG00000112335.15), PCBP1 (Accession #ENSG00000169564.7), NEAT1 (Accession #ENSG00000245532.9), CPEB4 (Accession #ENSG00000113742.14), FCHO2 (Accession #ENSG00000157107.14), TPI1 (Accession #ENSG00000111669.15), ATF4 (Accession #ENSG00000128272.17), CAPRIN1 (Accession #ENSG00000135387.21), CFLAR (Accession #ENSG00000003402.20), PEA15 (Accession #ENSG00000162734.13), NCOA4 (Accession #ENSG00000266412.6), BANK1 (Accession #ENSG00000153064.12), RPA1 (Accession #ENSG00000132383.12), SERF2 (Accession #ENSG00000140264.21), AKT3 (Accession #ENSG00000117020.19), ZYX (Accession #ENSG00000159840.16), CHMP4B (Accession #ENSG00000101421.4), CASP4 (Accession #ENSG00000196954.14), NCF2 (Accession #ENSG00000116701.15), ADAM10 (Accession #ENSG00000137845.15), DCUN1D1 (Accession #ENSG00000043093.15), AP1B1 (Accession #ENSG00000100280.17), WWP1 (Accession #ENSG00000123124.14), ZNF451 (Accession #ENSG00000112200.17), ZFHX3 (Accession #ENSG00000140836.17), LYPLAL1 (Accession #ENSG00000143353.12), HELZ (Accession #ENSG00000198265.12), GAS5 (Accession #ENSG00000234741.8), MAGI2-AS3 (Accession #ENSG00000234456.9), SAMD14 (Accession #ENSG00000167100.15), and GOSR1 (Accession #ENSG00000108587.16). In some embodiments, the biomarkers are selected from FKBP5, CCND1, GBP5, KAT6B, FCER1G, Clorf115, and LBH. In some embodiments, the biomarkers are selected from CST3, CCND1, LTA4H, STAT1, TMCC2, GBP5, and KAT6B. In some embodiments, the biomarkers are selected from GBP5, GBP1, GBP2, and GBP4. In some embodiments, the biomarkers are selected from GBP1, DUSP3, GBP2, GPX4, LPP, BNIP3L, KAT6B, LCP2, YOD1, FCHO2, UBXN7, FCN1, H2BC11, MBOAT2, FUT8, FGL2, and NAPA. In some embodiments, the biomarkers are selected from STAT1, LBH, LTA4H, SPTAN1, FCGR3A, PARP9, GLRX5, MS4A3, NFIA, CASP4, PARN, H3C2, and NDUFAF3.

In some embodiments, the panel of biomarkers comprises one or more genes selected from macrophage markers, neutrophil markers, interferon genes, or antimicrobial genes. There are a large number of commonly known and used macrophage markers. In some embodiments, the macrophage markers comprise of one or more genes selected from the group consisting of MARCO, SOCS3, FCGR1A, MPO, and C1QB.

Neutrophils can amount to as much as 70% of all leukocytes in the human body and are a major component of the immune system. There are a number of neutrophil markers that are commonly known and used in the art. Some common neutrophils are used to describe various neutrophil subpopulations depending on stages of neutrophil life cycle, including maturation, activation, migration, and various other neutrophil subsets, for example, Low-density neutrophils (LDN), PMN-MDSC (polymorphonuclear MDSC, also called granulocytic-MDSC), Proangiogenic neutrophils (PAN), and tumor-associated neutrophils (TAN). In some embodiments of the current disclosure, the neutrophil markers comprise of one or more genes selected from the group consisting of ELANE, FCGR3A, S100A8, S100A9, and ERG.

In some embodiments, the interferon genes comprise of one or more genes selected from the group consisting of IFI27L1, IFIT2, IFIT3, IFITM3, and IRF1.

In some embodiments, the antimicrobial genes comprise of one or more genes selected from the group consisting of AZU1, CTSG, DEFA4, STAT1, GBP1, GBP2, GBP4, GBP5, and GBP6.

In some embodiments, the panel of biomarkers comprises one or more genes selected from the group consisting of CST3, CCND1, LTA4H, STAT1, TMCC2, GBP5, KAT6B, LBH, FKBP5, Clorf115, FCER1G, DYSF, SMARCD3, VAMP5, FCGR3B, GPI, MPO, CREB5, and GBP2.

In some embodiments, the panel of biomarkers comprises GBP5. In some embodiments, the panel of biomarkers comprises GBP5 and one or more additional genes. In some embodiments, the panel of biomarkers comprises GBP5 and 2-8 additional genes. In some embodiments, the panel of biomarkers comprises GBP5 and one or more genes selected from CCND1, JADE1, ALB, NFIA, KAT6B, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, CST3, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1. In some embodiments, the panel of biomarkers comprises GBP5 and one or more genes selected from LBH, KAT6B, FKBP5, CCND1, Clorf115, and FCER1G. In some embodiments, the panel of biomarkers comprises GBP5 and one or more genes selected from DYSF, SMARCD3, VAMP5, FCGR3B, GPI, MPO, CREB5, and GBP2.

In some embodiments, the panel of biomarkers comprises CST3. In some embodiments, the panel of biomarkers comprises CST3 and one or more additional genes. In some embodiments, the panel of biomarkers comprises CST3 and 2-8 additional genes. In some embodiments, the panel of biomarkers comprises CST3 and one or more genes selected from GBP5, CCND1, JADE1, ALB, NFIA, KAT6B, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises CCND1. In some embodiments, the panel of biomarkers comprises CCND1 and one or more additional genes. In some embodiments, the panel of biomarkers comprises CCND1 and 2-8 additional genes. In some embodiments, the panel of biomarkers comprises CCND1 and one or more genes selected from GBP5, CST3, JADE1, ALB, NFIA, KAT6B, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises LTA4H. In some embodiments, the panel of biomarkers comprises LTA4H and one or more additional genes. In some embodiments, the panel of biomarkers comprises LTA4H and 2-8 additional genes. In some embodiments, the panel of biomarkers comprises LTA4H and one or more genes selected from GBP5, CST3, CCND1, JADE1, ALB, NFIA, KAT6B, H19, PURA, FKBP5, FCGR3B, LPP, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises STAT1. In some embodiments, the panel of biomarkers comprises STAT1 and one or more additional genes. In some embodiments, the panel of biomarkers comprises STAT1 and 2-8 additional genes. In some embodiments, the panel of biomarkers comprises STAT1 and one or more genes selected from GBP5, CST3, CCND1, JADE1, ALB, NFIA, KAT6B, H19, PURA, FKBP5, FCGR3B, LPP, LTA4H, S100A8, S100A9, TMCC2, LBH, MEA1, GBP4, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises TMCC2. In some embodiments, the panel of biomarkers comprises TMCC2 and one or more additional genes. In some embodiments, the panel of biomarkers comprises TMCC2 and 2-8 additional genes. In some embodiments, the panel of biomarkers comprises TMCC2 and one or more genes selected from GBP5, CST3, CCND1, JADE1, ALB, NFIA, KAT6B, H19, PURA, FKBP5, FCGR3B, LPP, LTA4H, S100A8, S100A9, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises KAT6B. In some embodiments, the panel of biomarkers comprises KAT6B and one or more additional genes. In some embodiments, the panel of biomarkers comprises KAT6B and 2-8 additional genes. In some embodiments, the panel of biomarkers comprises KAT6B and one or more genes selected from GBP5, CST3, CCND1, JADE1, ALB, NFIA, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, determining a tuberculosis disease status in the subject based on the profile of the panel of biomarkers comprises comparing the profile of the panel of biomarkers in the cfRNA from the biological sample to a reference profile of the panel of biomarkers. In some embodiments, determining a tuberculosis disease status in the subject based on the profile of the panel of biomarkers comprises comparing the levels of RNAs corresponding to the biomarkers in the panel to levels of RNAs corresponding to the biomarkers in the reference profile.

In some embodiments, a tuberculosis disease status comprises a tuberculosis free status, a latent tuberculosis status, an active tuberculosis status, or a tuberculosis progressing status. As used herein, “tuberculosis disease status” refers to TB related conditions or progression of TB disease. Not all subjects infected with Mycobacterium tuberculosis progress to a TB disease state. For example, latent TB infection refers to the TB condition where M. tuberculosis bacteria live in a subject, but the bacteria do not reproduce, or are inactive. In TB disease, the M. tuberculosis bacteria in the subject are active and reproducing in the subject.

In some embodiments, the biological sample is a plasma sample. In some embodiments, the biological sample is a serum sample. In some embodiments, the subject is a mammal. In some embodiments, the subject is a human. In some embodiments, the profile of the panel of biomarkers comprises subject-specific gene expression of the biomarkers in the panel. In some embodiments, the profile of the panel of biomarkers comprises a score determined based on the levels of RNAs corresponding to the biomarkers in the panel using a trained classification model, and wherein the score correlates with disease status.

In some embodiments, the cell-free RNA is substantially free of contaminant RNA. “Contaminant RNA” refers to RNA that is not from the subject. The contaminant RNA could be RNA from other bacteria in the subject. cfRNA that is substantially free of contaminant RNA means that the signature transcripts are from the subject or host RNA, rather than from RNA produced by a foreign pathogen, such as Mycobacterium tuberculosis bacterium.

In some embodiments, the tuberculosis disease status determined is used in the triaging of the subject. In some embodiments, the tuberculosis disease status determined is used in determining treatment for the subject. In some embodiments, the tuberculosis disease status determined is used to monitor treatment of the subject. In some embodiments, the tuberculosis disease status determined is used to monitor efficacy of a treatment administered to the subject.

In some embodiments, test performance of the trained classification model is measured through one or more of accuracy, sensitivity, specificity, and/or area under the receiver operating characteristic curve (ROC-AUC).

As used herein, “test accuracy” or “accuracy” provides the final generalization power. In some embodiments, the tuberculosis disease status is determined at a greater than 80% accuracy. In some embodiments, the tuberculosis disease status is determined at a greater than 85% accuracy. In some embodiments, the tuberculosis disease status is determined at a greater than 86% accuracy. In some embodiments, the tuberculosis disease status is determined at a greater than 87% accuracy. In some embodiments, the tuberculosis disease status is determined at a greater than 88% accuracy. In some embodiments, the tuberculosis disease status is determined at a greater than 89% accuracy. In some embodiments, the tuberculosis disease status is determined at a greater than 90% accuracy. In some embodiments, the tuberculosis disease status is determined at a greater than 91% accuracy. In some embodiments, the tuberculosis disease status is determined at a greater than 92% accuracy. In some embodiments, the tuberculosis disease status is determined at a greater than 93% accuracy. In some embodiments, the tuberculosis disease status is determined at a greater than 94% accuracy. In some embodiments, the tuberculosis disease status is determined at a greater than 95% accuracy.

As used herein, “sensitivity” describes the probability of a positive test result, conditioned on the individual truly being positive. Sensitivity is determined by the number of true positives divided by the sum of true positives and false negatives. As such, a test which reliably detects the presence of a condition, resulting in a high number of true positives and low number of false negatives, will have a high sensitivity. In some embodiments, the tuberculosis disease status is determined at a greater than 80% sensitivity. In some embodiments, the tuberculosis disease status is determined at a greater than 85% sensitivity. In some embodiments, the tuberculosis disease status is determined at a greater than 86% sensitivity. In some embodiments, the tuberculosis disease status is determined at a greater than 87% sensitivity. In some embodiments, the tuberculosis disease status is determined at a greater than 88% sensitivity. In some embodiments, the tuberculosis disease status is determined at a greater than 89% sensitivity. In some embodiments, the tuberculosis disease status is determined at a greater than 90% sensitivity. In some embodiments, the tuberculosis disease status is determined at a greater than 91% sensitivity. In some embodiments, the tuberculosis disease status is determined at a greater than 92% sensitivity. In some embodiments, the tuberculosis disease status is determined at a greater than 93% sensitivity. In some embodiments, the tuberculosis disease status is determined at a greater than 94% sensitivity. In some embodiments, the tuberculosis disease status is determined at a greater than 95% sensitivity.

As used herein, “specificity” refers to the probability of a negative test result, conditioned on the individual truly being negative. Specificity is determined by the number of true negatives divided by the sum of true negatives and false positives. As such, a test which reliably excludes individuals who do not have the condition, resulting in a high number of true negatives and low number of false positives, will have a high specificity. In some embodiments, the tuberculosis disease status is determined at a greater than 70% specificity. In some embodiments, the tuberculosis disease status is determined at a greater than 75% specificity. In some embodiments, the tuberculosis disease status is determined at a greater than 80% specificity. In some embodiments, the tuberculosis disease status is determined at a greater than 81% specificity. In some embodiments, the tuberculosis disease status is determined at a greater than 82% specificity. In some embodiments, the tuberculosis disease status is determined at a greater than 83% specificity. In some embodiments, the tuberculosis disease status is determined at a greater than 84% specificity. In some embodiments, the tuberculosis disease status is determined at a greater than 85% specificity. In some embodiments, the tuberculosis disease status is determined at a greater than 86% specificity. In some embodiments, the tuberculosis disease status is determined at a greater than 87% specificity. In some embodiments, the tuberculosis disease status is determined at a greater than 88% specificity. In some embodiments, the tuberculosis disease status is determined at a greater than 89% specificity. In some embodiments, the tuberculosis disease status is determined at a greater than 90% specificity. In some embodiments, the tuberculosis disease status is determined at a greater than 91% specificity. In some embodiments, the tuberculosis disease status is determined at a greater than 92% specificity. In some embodiments, the tuberculosis disease status is determined at a greater than 93% specificity. In some embodiments, the tuberculosis disease status is determined at a greater than 94% specificity. In some embodiments, the tuberculosis disease status is determined at a greater than 95% specificity.

In some embodiments, the trained classification model comprises one or more models selected from the group consisting of generalized linear models with Ridge and LASSO feature selection (GLMNETRIDGE and GLMNETLASSO), support vector machines with linear and radial basis function kernel (SVMLin and SVMRAD), random forest (RF), random forest ExtraTrees (EXTRATREES), neural networks (NNET), linear discriminant analysis (LDA), nearest shrunken centroids (PAM), C5.0 (C5), k-nearest neighbors (KNN), naive bayes (NB), CART (RPART), generalized linear model (GLM), and greedy forward search algorithm (GFS).

In some embodiments, the method further comprises step d) treating the subject based on the tuberculosis disease status. In some embodiments, step d) treating the subject based on the tuberculosis disease comprises treating the subject with an antibiotic. Treatments for TB are known in the art, for example isoniazid, rifampin, rifapentine, moxifloxacin, pyrazinamide, ethambutol and streptomycin are all used in the treatment of TB.

An aspect of the disclosure is directed to a method for establishing a panel of biomarkers that correlates with tuberculosis disease status, comprising:
a) obtaining cell-free RNA from a biological sample of each of a group of subjects, wherein the biological sample is a plasma sample or a serum sample, and wherein the group of subjects comprise subjects positive for tuberculosis and subjects negative for tuberculosis;
b) detecting levels of cell-free RNAs of a plurality of genes in the subjects;
c) dividing the subjects into a training set and a testing set, wherein each set comprises subjects positive for tuberculosis and subjects negative for tuberculosis;
d) based on levels of cell-free RNAs of a plurality of genes in the training set, selecting a panel of genes, wherein the selecting comprises:
   i) selecting differentially abundant genes based on a differential analysis; and
   ii) training a classification model using the levels of cell-free RNAs of the selected genes; thereby determining a classification score for optimal classifier performance in an ROC (Receiver Operating Characteristics) analysis; and
e) testing the trained classification model by applying the trained model and the classification score to the cell-free RNA levels of the testing set, and determining the panel of genes as the panel of biomarkers that correlates with tuberculosis disease status wherein the trained classification model provides differentiation in the testing between subjects positive for tuberculosis and subjects negative for tuberculosis with at least 85% sensitivity and at least 65% specificity.

As used herein, “training set” is a collection of samples or a group of subject samples used to “teach” or “train” the machine learning model. The model uses a training set to understand the patterns and relationships within the data, thereby learning to make predictions or decisions without being explicitly programmed to perform a specific task.

In some embodiments, the group of subject samples include at least 50 subject samples positive for TB, and at least 50 subject samples negative for TB. In some embodiments, the group of subject samples include at least 75 subject samples positive for TB, and at least 75 subject samples negative for TB. In some embodiments, the group of subject samples include at least 85 subject samples positive for TB, and at least 85 subject samples negative for TB. In some embodiments, the group of subject samples include at least 100 subject samples positive for TB, and at least 100 subject samples negative for TB. The group of subject samples does not need an equal number of subject samples positive for TB and subject samples for TB. For example, in some embodiments, the group of subject samples could include at least 75 subject samples positive for TB and at least 70 subject samples negative for TB.

In some embodiments, step (c) of the method for establishing a panel of biomarkers that correlates with tuberculosis disease status further comprises dividing the subjects into a training set, a validation set, and a testing set, wherein each set comprises subjects positive for tuberculosis and subjects negative for tuberculosis. As used herein, “validation set” refers to a collection of samples used to estimate the accuracy of the machine learning model. In some embodiments, the validation set is used as an estimation of how well the model might perform beyond the scope of the training set and test set. In some embodiments, certain panels of genes can have high train-test performance and have subpar validation performance.

The sensitivity and specificity of the model are not necessarily linked, meaning sensitivity and specificity may each change without the other aspect changing. For example, in some embodiments, the trained classification model provides differentiation in the testing between subjects positive for tuberculosis and subjects negative for tuberculosis with at least 85% sensitivity and at least 65% specificity; while in other embodiments, the trained classification model provides differentiation in the testing between subjects positive for tuberculosis and subjects negative for tuberculosis with at least 85% sensitivity and at least 70% specificity. Yet, in other embodiments, the trained classification model provides differentiation in the testing between subjects positive for tuberculosis and subjects negative for tuberculosis with at least 87% sensitivity and at least 65% specificity. In some embodiments, the differentially abundant genes are selected by removing genes of low count and genes having an adjusted p-value greater than 0.05.

In some embodiments, the classification model comprises one or more models selected from the group consisting of generalized linear models with Ridge and LASSO feature selection (GLMNETRIDGE and GLMNETLASSO), support vector machines with linear and radial basis function kernel (SVMLin and SVMRAD), random forest (RF), random forest ExtraTrees (EXTRATREES), neural networks (NNET), linear discriminant analysis (LDA), nearest shrunken centroids (PAM), C5.0 (C5), k-nearest neighbors (KNN), naive bayes (NB), CART (RPART), generalized linear model (GLM), and greedy forward search algorithm (GFS).

In some embodiments, the panel of biomarkers comprises at least 1 gene. In some embodiments, the panel of biomarkers comprises one gene, and that gene is selected from GBP5, CCND1, JADE1, ALB, NFIA, KAT6B, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, CST3, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises GBP5. In some embodiments, the panel of biomarkers comprises GBP5 and at least one or more additional marker. In some embodiments, the panel of biomarkers comprises GBP5 and 2-8 additional genes. In some embodiments, the at least one additional marker is selected from CCND1, JADE1, ALB, NFIA, KAT6B, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, CST3, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1. In some embodiments, the panel of biomarkers comprises GBP5 and one or more genes selected from LBH, KAT6B, FKBP5, CCND1, Clorf115, and FCER1G. In some embodiments, the panel of biomarkers comprises GBP5 and one or more genes selected from DYSF, SMARCD3, VAMP5, FCGR3B, GPI, MPO, CREB5, and GBP2.

In some embodiments, the panel of biomarkers comprises CST3. In some embodiments, the panel of biomarkers comprises CST3 and one or more additional genes. In some embodiments, the panel of biomarkers comprises CST3 and 2-8 additional genes. In some embodiments, the panel of biomarkers comprises CST3 and one or more genes selected from GBP5, CCND1, JADE1, ALB, NFIA, KAT6B, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises CCND1. In some embodiments, the panel of biomarkers comprises CCND1 and one or more additional genes. In some embodiments, the panel of biomarkers comprises CCND1 and 2-8 additional genes. In some embodiments, the panel of biomarkers comprises CCND1 and one or more genes selected from GBP5, CST3, JADE1, ALB, NFIA, KAT6B, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises LTA4H. In some embodiments, the panel of biomarkers comprises LTA4H and one or more additional genes. In some embodiments, the panel of biomarkers comprises LTA4H and 2-8 additional genes. In some embodiments, the panel of biomarkers comprises LTA4H and one or more genes selected from GBP5, CST3, CCND1, JADE1, ALB, NFIA, KAT6B, H19, PURA, FKBP5, FCGR3B, LPP, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises STAT1. In some embodiments, the panel of biomarkers comprises STAT1 and one or more additional genes. In some embodiments, the panel of biomarkers comprises STAT1 and 2-8 additional genes. In some embodiments, the panel of biomarkers comprises STAT1 and one or more genes selected from GBP5, CST3, CCND1, JADE1, ALB, NFIA, KAT6B, H19, PURA, FKBP5, FCGR3B, LPP, LTA4H, S100A8, S100A9, TMCC2, LBH, MEA1, GBP4, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises TMCC2. In some embodiments, the panel of biomarkers comprises TMCC2 and one or more additional genes. In some embodiments, the panel of biomarkers comprises TMCC2 and 2-8 additional genes. In some embodiments, the panel of biomarkers comprises TMCC2 and one or more genes selected from GBP5, CST3, CCND1, JADE1, ALB, NFIA, KAT6B, H19, PURA, FKBP5, FCGR3B, LPP, LTA4H, S100A8, S100A9, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the panel of biomarkers comprises KAT6B. In some embodiments, the panel of biomarkers comprises KAT6B and one or more additional genes. In some embodiments, the panel of biomarkers comprises KAT6B and 2-8 additional genes. In some embodiments, the panel of biomarkers comprises KAT6B and one or more genes selected from GBP5, CST3, CCND1, JADE1, ALB, NFIA, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

In some embodiments, the trained classification model provides differentiation in the testing between subjects positive for tuberculosis and subjects negative for tuberculosis with at least 80% sensitivity. In some embodiments, the trained classification model provides differentiation in the testing between subjects positive for tuberculosis and subjects negative for tuberculosis with at least 60% specificity. In some embodiments, the trained classification model provides differentiation in the testing between subjects positive for tuberculosis and subjects negative for tuberculosis with at least 90% sensitivity and at least 70% specificity.

In some embodiments, when the trained classification model does not provide differentiation in the testing between subjects positive for tuberculosis and subjects negative for tuberculosis with at least 85% sensitivity and at least 65% specificity. In such an event, the method further comprises one or a combination of steps:
f) selecting a different modeling technique;
g) adjusting the hyperparameters for training the model;
h) changing the initial filtering steps and how Differential abundance analysis is performed; and/or
i) adjusting the classifier score threshold based on the training set.

In some embodiments, step (f) comprises selecting a different modeling technique wherein the classification model comprises one or more models selected from the group consisting of generalized linear models with Ridge and LASSO feature selection (GLMNETRIDGE and GLMNETLASSO), support vector machines with linear and radial basis function kernel (SVMLin and SVMRAD), random forest (RF), random forest ExtraTrees (EXTRATREES), neural networks (NNET), linear discriminant analysis (LDA), nearest shrunken centroids (PAM), C5.0 (C5), k-nearest neighbors (KNN), naive bayes (NB), CART (RPART), generalized linear model (GLM), and greedy forward search algorithm (GFS).

In some embodiments, step (g) comprises adjusting the hyperparameters for training the model.

In some embodiments, step (h) comprises changing the initial filtering steps and how Differential abundance analysis is performed. Such an adjustment changes which genes are input into the machine learning models.

In some embodiments, step (i) comprises adjusting the classifier score threshold based on the training set. Adjusting the classifier score based on the training set allows optimization of the threshold for sensitivity or specificity.

### EXAMPLES

### Example 1: Materials and Methods

### Ethics Statement

All subjects provided written informed consent and all experiments were performed in accordance with relevant guidelines and regulations. The protocols for this study were approved locally at each site by Institutional Review Boards at Cornell University (protocols IRB0145569, 1902008555); UCSF (protocol 20-32670); Heidelberg University (5-539/2020); the Makerere University School of Medicine (protocol 2017-020); Vietnam National Lung Hospital (protocol 566/2020/NCKH), and De La Salle Medical and Health Sciences Institute (protocol 2020-33-02-A).

### Sample Collection

Plasma samples were collected as part of three independent clinical studies, Evaluation of Novel Diagnostics for Tuberculosis (END TB), the Rapid Research in Diagnostics Development for TB Network (R2D2), and TB2. The studies enrolled adults with cough ≥2 weeks identified at outpatient clinics in Uganda (END TB and TB2) and in Uganda, Vietnam, and the Philippines (R2D2).

Peripheral blood samples from individuals enrolled in the END TB and TB2 studies in Uganda were collected in Streck Cell-Free DNA blood collection tubes (Streck, 230257). Plasma was separated by centrifugation at 1,600×g for 10 minutes at ambient temperature. The remaining cellular debris was removed by an additional centrifugation step at 16,000×g for 10 minutes at ambient temperature. Plasma was stored in 1 mL aliquots at −80° C.

Plasma samples from individuals enrolled in the R2D2 study sites in Uganda, Vietnam, and the Philippines were collected in K2EDTA blood collection tubes (BD Diagnostics, 366643). Plasma was separated by centrifugation at 1,600×g for 10 minutes at ambient temperature in a horizontal rotor (swing out head). Plasma was similarly stored in 1 mL aliquots at −80° C.

Matched whole blood samples from individuals enrolled in the TB2 study in Uganda were collected in PAXgene Blood RNA tubes (Qiagen, 762165). The 2.5-mL PAXgene tubes were stored at −80° C.

Plasma cfRNA Isolation and Library Preparation

Plasma samples were received on dry ice and stored at −80° C. until processed. Prior to cfRNA extraction, plasma samples were thawed at room temperature and centrifuged at 1300×g for 10 minutes at 4° C. cfRNA was extracted from plasma (300-1,000 l) using the Norgen Plasma/Serum Circulating and Exosomal RNA Purification Mini Kit (Norgen, 51000). Extracted RNA was DNase treated with 14 μl of 10 μl DNase Turbo Buffer (Invitrogen, AM2238), 3 μl DNase Turbo (Invitrogen, AM2238), and 1 μl Baseline Zero DNase (Lucigen-Epicenter, DB0715K) for 30 minutes at 37° C., then concentrated into 12 μl using the Zymo RNA Clean and Concentrated Kit (Zymo, R1015).

Sequencing libraries were prepared from 8 μl of concentrated RNA using the Takara SMARTer® Stranded Total RNA-Seq Kit v3—Pico Input Mammalian (Takara, 634485) and barcoded using the SMARTer® RNA Unique Dual Index Kit (Takara, 634451). Library concentration was quantified using the Qubit™ 3.0 Fluorometer (Invitrogen, Q33216) with the dsDNA HS Assay Kit (Invitrogen, Q32854). Libraries were quality-controlled using the Agilent Fragment Analyzer 5200 (Agilent, M5310AA) with the HS NGS Fragment Kit (Agilent, DNF-474-0500) and pooled to equal concentrations. Each pool was sequenced using both the Illumina NextSeq 500/550 platform (paired-end, 150 bp) and the Illumina NextSeq 2000 platform (paired-end, 100 bp).

### Whole Blood RNA Isolation and Library Preparation

Whole blood samples were received on dry ice and stored at −80° C. until processed. Prior to RNA extraction, whole blood samples were thawed at 37° C. RNA was extracted form whole blood samples (700 μl) using the Quick-RNA Whole Blood Kit (Zymo, R1201) following manufacturer's instructions. RNA was processed using the NEBNext Globin & rRNA Depletion Kit (Human/Mouse/Rat; NEB, E7750X) and libraries were created using the NEBNext Ultra II Directional RNA Library Prep Kit (NEB, E7760 and E7765) following manufacturer's specifications.

Sequencing libraries were quantified using the Qubit™ 3.0 Fluorometer (Invitrogen, Q33216) with the dsDNA HS Assay Kit (Invitrogen, Q32854). Libraries were quality-controlled using the Agilent Fragment Analyzer 5200 (Agilent, M5310AA) with the HS NGS Fragment Kit (Agilent, DNF-474-0500) and pooled to equal concentrations. Each pool was sequencing using the Illumina NextSeq 2000 platform (paired-end, 100 bp).

### Bioinformatic Processing and Sample Quality Filtering

Sequencing data was processed using a custom bioinformatics pipeline. Since pools sequenced on the Illumina NextSeq 2000 were optimized to produce paired-end, 61 bp reads, matched sequencing data from the Illumina NextSeq 500 were trimmed using Seqtk (v1.2). Samples were then quality filtered and trimmed using BBDuk (v38.90), aligned to the Gencode GRCh38 human reference genome (v38, primary assembly) using STAR³¹ (v2.7.0f, default parameters). Prior to feature quantification using featureCounts (v2.0.0), samples were deduplicated using Picard MarkDuplicates (v2.19.2). Mitochondrial, ribosomal, X, and Y chromosome genes were bioinformatically removed prior to analysis.

Samples were filtered on the basis of DNA contamination, rRNA contamination, total counts, and RNA degradation. DNA contamination was estimated by calculating the ratio of reads mapping to introns and exons. rRNA contamination was measured using SAMtools (v1.14). Total counts were calculated using featureCounts (v2.0.0). Degradation was estimated by calculating the 5′-3′ bias using Qualimap (v2.2.1).

Samples were removed from analysis if either the intron to exon ratio was greater than 3, if a sample had less than 75,000 total counts, or if the rRNA contamination, total counts, or 5′-3′ bias was greater than three standard deviations from the mean.

### Cell Type Deconvolution

Cell type deconvolution was performed using BayesPrism (v1.1) with the Tabula Sapiens single-cell RNA-seq atlas (Release 1) as a reference. Cells from the Tabula Sapiens atlas were grouped as previously described in Loy et al. Cell types with more than 100,000 unique molecular identifiers (UMIs) were included in the reference and subsampled to 300 cells using ScanPy (v1.8.1).

### Differential Abundance Analysis

Comparative analysis of differentially expressed genes was performed using a negative binomial model as implemented in DESeq2 (v1.34.0). Heatmaps were constructed using the pheatmap package in R (v1.0.12). Samples and genes were clustered using correlation-based hierarchical clustering. Canonical pathways, diseases and functions were analyzed using QIAGEN Ingenuity Pathway Analysis software (v73620684).

### Machine Learning and Model Training

Machine learning and model training was performed using R (v4.1.3) with the DESeq2 (v1.34.0), Caret (v6.0.90), and pROC (v1.18.0) packages. Sample metadata and count matrices were split 70/30 into a training set and a test set, controlling for disease status, HIV status, and cohort to minimize differences in the training and test datasets.

Features for model training were selected by filtering and differential abundance analysis. First, differential abundance analysis was performed on the raw training counts using DESeq2. Following DESeq2's internal filtering, 56,634 genes were retained. Then feature selection was conducted using the output from DESeq2. Genes were excluded that had a base-mean of less than 100 and a Benjamini-Hochberg adjusted p-value greater than 0.05. The remaining 113 genes were selected for model training.

Machine learning algorithms were trained using 5-fold cross validation and grid search hyperparameter tuning. Accuracy, sensitivity, specificity, and area under the receiver operating characteristic curve (ROC-AUC) were used to measure test performance. The classification models used were generalized linear models with Ridge and LASSO feature selection (GLMNETRIDGE and GLMNETLASSO), support vector machines with linear and radial basis function kernel (SVMLin and SVMRAD), random forest (RF), random forest ExtraTrees (EXTRATREES), neural networks (NNET), linear discriminant analysis (LDA), nearest shrunken centroids (PAM), C5.0 (C5), k-nearest neighbors (KNN), naive bayes (NB), CART (RPART), and generalized linear models (GLM). An additional model was trained and tested using a greedy forward search algorithm (GFS). Briefly, this algorithm iterates over the list of genes and evaluates each individual gene's discriminatory power by training a generalized linear model (GLM). The discriminatory power is evaluated based on a combined score which is calculated as: (AUC+Sensitivity+Specificity), in which sensitivity and specificity are calculated at the optimal youden threshold. The gene that results in the highest score is added to the model. At each subsequent iteration over the list of candidate genes, the algorithm will attempt to add a gene to the model in this way. The algorithm stops when there is no gene in the list that can increase the score by more than 0.01.

An additional model was trained and tested using a greedy forward search algorithm. Briefly, the algorithm starts with a single gene that provides the most discriminatory power using a tuberculosis score. The tuberculosis score was calculated as:

$$Tuberculosis score = < {log}_{2} ( CPM + 1 ) {>}_{u p regulated} - < {log}_{2} ( CPM + 1 ) {>}_{d ownregulated} ,$$

where CPM is counts per million obtained using the cpm function (edgeR v3.36.0) on raw counts. At each subsequent step, an additional gene is added and the tuberculosis score is recalculated. The combination of genes at each step that provides the greatest increase in AUC becomes the basis for the next iteration. The search stops when no gene addition increases the AUC.

### Quantification and Statistical Analyses

All statistical analyses were performed using R (v4.1.0). Statistical significance was tested using Wilcoxon signed-rank tests and Mann-Whitney U tests in a two-sided manner, unless otherwise stated. Boxes in the boxplots indicate the 25th and 75th percentiles, the band in the box represents the median, and whiskers extend to 1.5×interquartile range of the hinge. All sequencing data was aligned to the GRCh38 Gencode v38 Primary Assembly and features were counted using the GRCH38 Gencode v38 Primary Assembly Annotation.

### Example 2: Plasma Cell-Free RNA Signatures of Tuberculosis

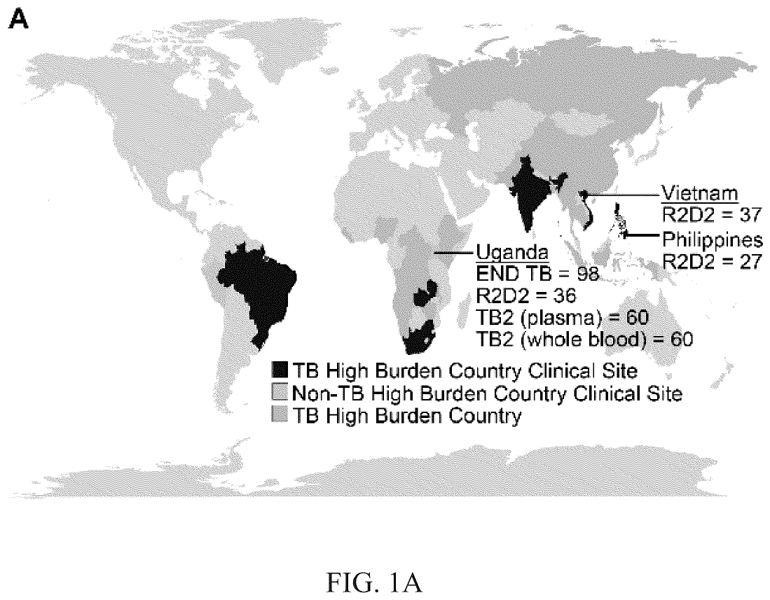

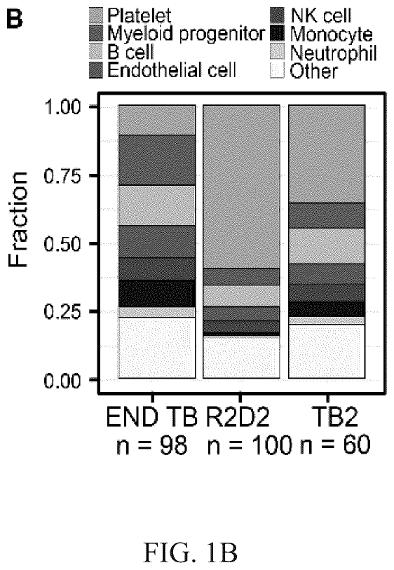

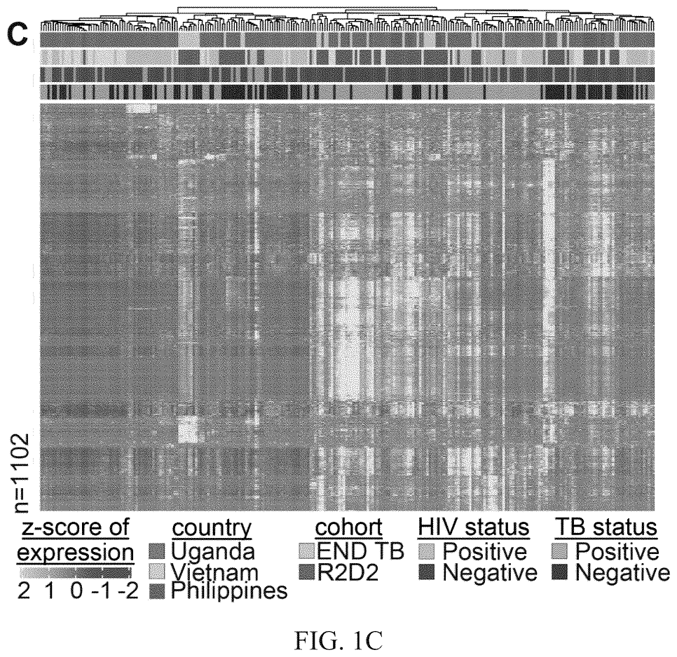

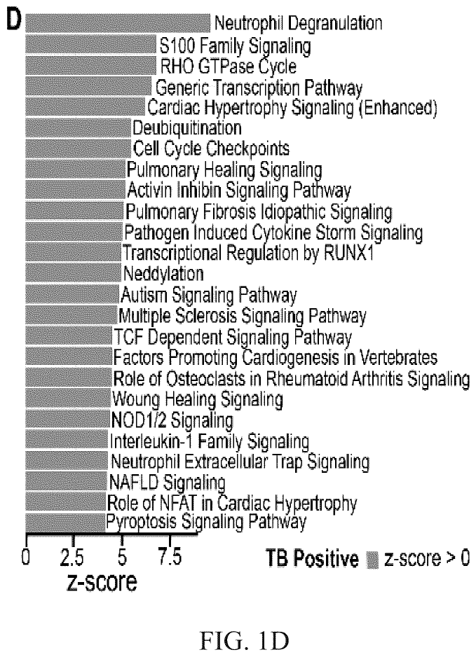

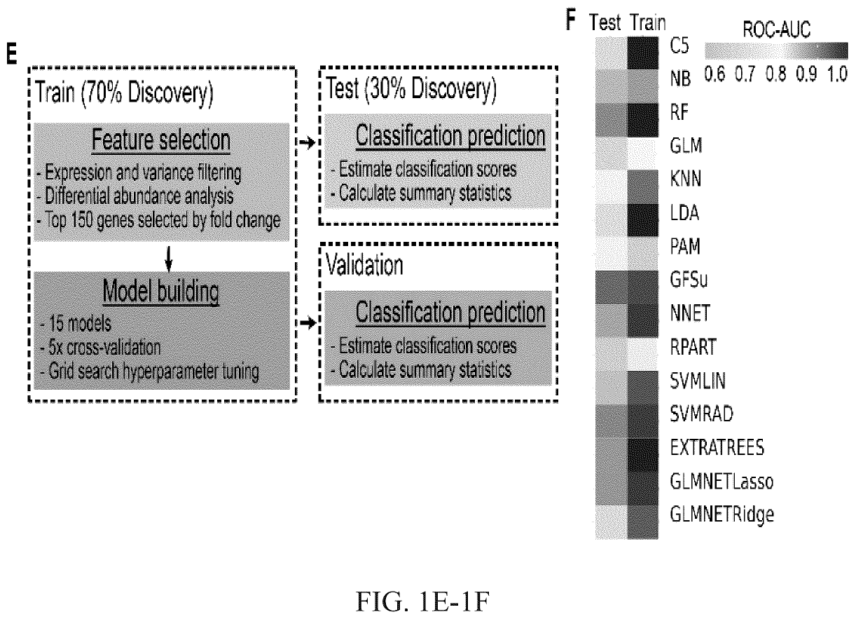

258 plasma samples were analyzed from people with a cough lasting at least two weeks who were enrolled in three independent clinical studies (END TB, R2D2, and TB2) at outpatient clinics in Uganda, Vietnam, and the Philippines (Table 1 and FIG. 1A). Individuals included in the “TB positive” group were required to have 1) a positive Xpert MTB/RIF Ultra on sputum, urine, or contaminated Mycobacterial Growth Indicator Tube (MGIT) specimen; 2) a positive sputum MGIT or solid culture; or, 3) two trace Xpert Ultra results on sputum or contaminated MGIT. All other individuals (“TB negative” group) had at least one negative Xpert Ultra result and two negative cultures in MGIT or solid media. Of the 258 plasma samples analyzed in our study, 145 (56.5%) were from individuals diagnosed with microbiologically confirmed TB and 39 (15%) were from HIV-negative individuals. Next-generation sequencing was used to profile the RNA in these blood samples, yielding a mean of 31,526,069 reads per sample in plasma.

TABLE 1

| | END TB | | | | R2D2 | | | | TB2 | | | |
|---|---|---|---|---|---|---|---|---|---|---|---|---|
| | TB | | TB | | TB | | TB | | TB | | TB | |
| | Positive | | Negative | | Positive | | Negative | | Positive | | Negative | |
| n | 60 | | 38 | | 50 | | 50 | | 35 | | 25 | |
| Age, median | 29 | (25-37) | 30 | (25-38.8) | 36 | (27-51) | 47.5 | (32-58.8) | 30 | (26-35) | 34 | (30-42) |
| (IQR) | | | | | | | | | | | | |
| Females, n | 16 | (26.6) | 21 | (55.3) | 17 | (34) | 24 | (48) | 10 | (26.6) | 8 | (32) |
| (%) | | | | | | | | | | | | |
| HIV positive, n | 8 | (13.3) | 10 | (26.3) | 3 | (6) | 8 | (16) | 7 | (20) | 3 | (12) |
| (%) | | | | | | | | | | | | |
| Prior TB, n | 4 | (6.7) | 9 | (23.7) | 12 | (24) | 6 | (12) | 5 | (14.3) | 2 | (8) |
| (%) | | | | | | | | | | | | |

A reference-based deconvolution algorithm (BayesPrism) was used to determine the cell-types-of-origin of the cfRNA in the samples. This algorithm uses a reference dataset, in this case the Tabula Sapiens single-cell RNA-seq atlas, and bayesian inference to identify the cell types from which the cfRNA in the samples originated. The plasma cfRNA in individuals with respiratory symptoms was primarily derived from platelets, myeloid progenitor cells, B cells, endothelial cells, natural killer cells, monocytes, and neutrophils (FIG. 1B). Batch effects were observed in the platelet contribution. There was a significant difference in the platelet contribution between all three cohorts (mean fraction: 0.11, 0.60, 0.36 for END TB, R2D2, and TB2, respectively). These differences were due to the different sample collection tubes and plasma collection procedures used by the END TB, R2D2, and TB2 studies

Next, a differential abundance analysis was performed using DESeq2 to gain insight into the host response to TB and to identify potential diagnostic biomarkers. Using a Benjamini-Hochberg adjusted p-value cutoff of <0.05, 1102 differentially abundant genes were identified between TB positive and negative groups (FIG. 1C). TB was associated with elevated levels of macrophage markers (FCGR1A, MPO, C1QB), neutrophil markers (ELANE, FCGR3A, S100A8, S100A9, ERG), interferon genes (IFIT2, IFIT3, IFITM3, IRF1), and antimicrobial genes (AZU1, CTSG, DEFA4, STAT1, GBP1, GBP2, GBP4, GBP5) compared to the TB negative group. Elevated levels of lung-specific markers (SLC34A2, SFTPA2) were observed in individuals with TB, providing insight into ongoing pathogenic processes. Ingenuity Pathway Analysis was performed to confirm relevance of these findings (FIG. 1D). The top canonical pathways enriched in TB (ranked by log(p-value)>1.5 and |z-score|>=4) included pyroptosis signaling, neutrophil degranulation, and pathogen-induced cytokine storm signaling pathways.

### Example 3: Evaluating Machine Learning Algorithms for Gene-Expression-Based TB Diagnostics

The results from the differential expression analysis, including enrichment of clinically relevant pathways and satisfactory separation of sample groups using correlation-based clustering, suggested that differentially abundant genes in plasma cfRNA could be used to develop triage tests and diagnostic assays for TB. To test this, the samples were split into a “discovery” set (END TB and R2D2) and a “validation” set (TB2). The discovery cohort samples were first divided into a training set (70%) and a test set (30%), to ensure that disease status, HIV-status, and cohort were equally represented (FIG. 1E). Then, differential abundance analysis was performed on the training set using DESeq2 to identify relevant candidate genes. Next, the DESeq2 output was used to perform feature selection on these candidate genes by removing genes with a Benjamini-Hochberg adjusted p-value greater than 0.05 and a base-mean less than 100 (Methods). The raw counts were normalized using variance stabilizing transformation (VST). The normalized counts from the training set were subset to only include genes from feature selection and then were used to train 15 machine learning classification models. The classification score threshold was determined by the optimal youden threshold calculated on the training set.

Many of the models performed well, with 7/15 models having a train and test area under the receiver operating characteristic curve (ROC-AUC)>0.85 (FIGS. 1F-1G). However, some models, such as the C5 classification model (C5) and linear discriminant analysis (LDA), suffered from overfitting (perfect training set performance but poor test performance). The poor performance of GLM may be due to the lack of regularization, since the performance of the generalized linear models with Ridge and LASSO feature selection (GLMNETRIDGE and GLMNETLASSO) were on par with the other models, suggesting that there may be several highly correlated significant features. This high collinearity may also explain the poor performance of LDA.

### Example 4: 7-Gene Biomarker Panel Surpasses Minimal Guidelines for a Non-Sputum-Based Triage Test

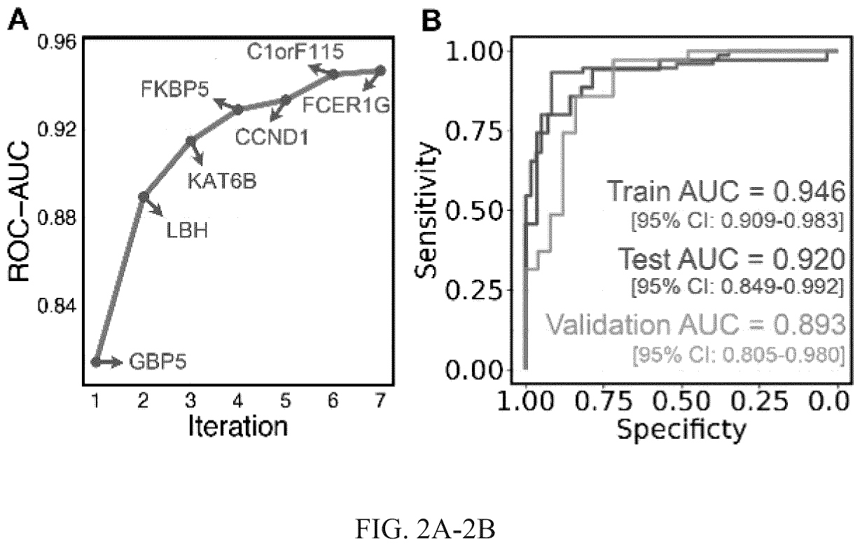

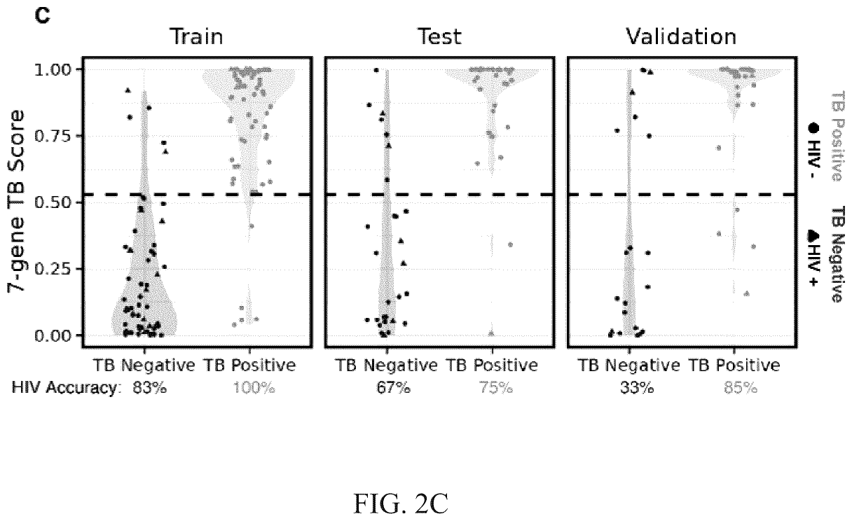

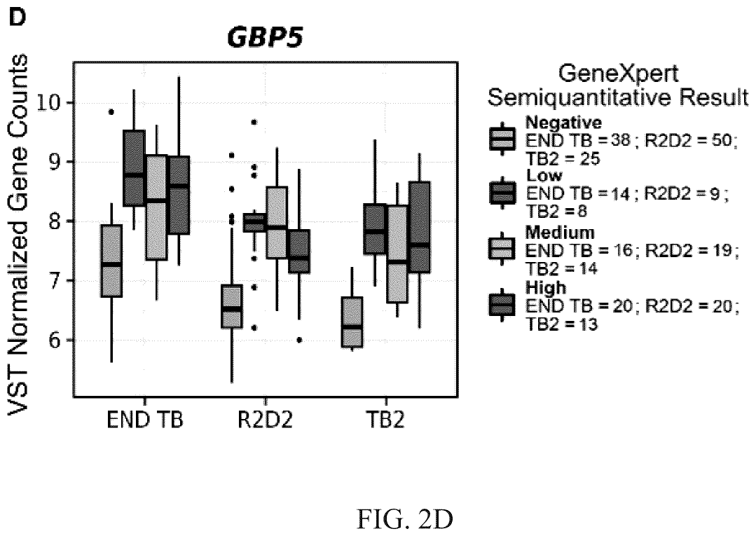

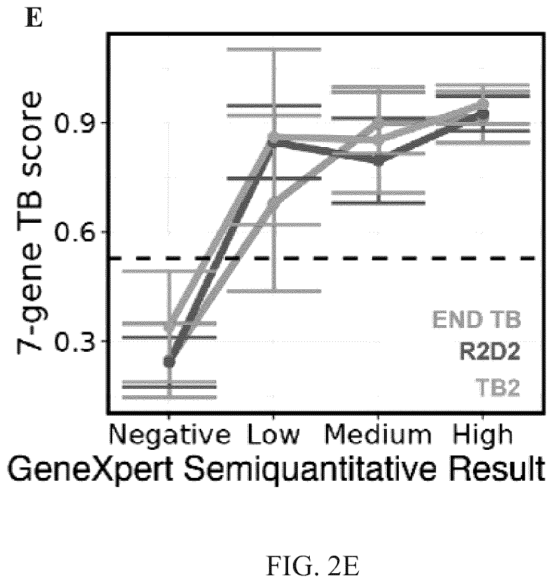

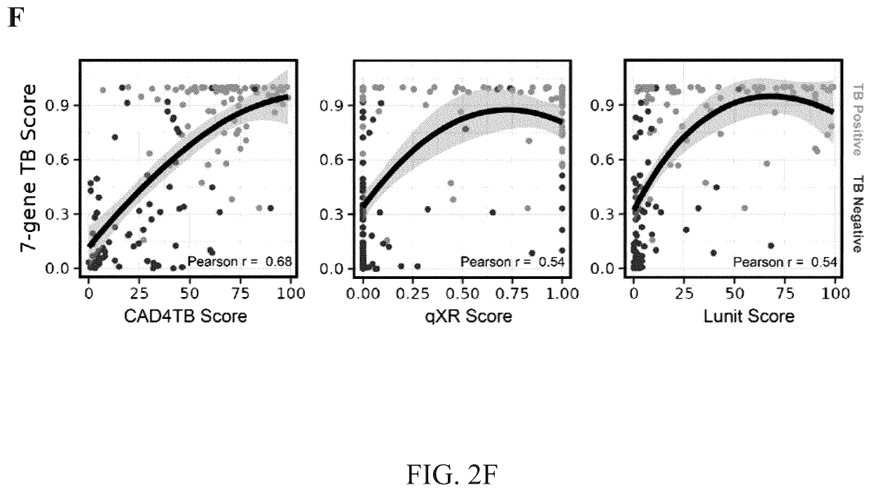

After evaluating the performance of the different machine learning models, the candidate biomarker panel was based off the greedy forward search results because it was the most consistent across training and test and the highest performing model on the test set. The greedy forward search algorithm is initiated by choosing the gene with the most discriminatory power in terms of a TB score, which is determined by ROC-AUC value. In each subsequent iteration, the algorithm adds the gene that produces the greatest increase in a score which is calculated as: ROC-AUC+Sensitivity+Specificity. This yielded a 7-gene signature consisting of guanylate binding protein 5 (GBP5), limb bud-heart (LBH), lysine acetyltransferase 6B (KAT6B), FK506 binding protein 5 (FKBP5), cyclin D1 (CCND1), chromosome 1 open reading frame 115 (Clorf115), high affinity immunoglobin epsilon receptor subunit gamma (FCER1G) (FIG. 2A).

The utility of the 7-gene signature as a diagnostic or triage tool was evaluated using ROC analysis on the training and test sets. The WHO TPPs provide the minimal and optimal characteristics of a target product, where the minimal thresholds indicate the lowest acceptable standards, and the optimal criteria outline the ideal target profile. For a non-sputum-based diagnostic test, the overall minimal and optimal sensitivities are 65% and 80% with a specificity of 98%; for a non-sputum-based triage test, the optimal requirements are 95% sensitivity and 80% specificity, while the minimal criteria are 90% sensitivity and 70% specificity. The discriminatory power of the signature was high in both sets (training set ROC-AUC=0.946 vs test set ROC-AUC=0.920; FIG. 2B). In the training set, the TB score discriminated between TB positive and negative groups with 92.6% accuracy, 93.3% sensitivity, and 91.7% specificity. In the test set, the TB score discriminated between TB positive and negative groups with 85.7% accuracy, 94.3% sensitivity, and 75.0% specificity (FIG. 2C). This biomarker panel exceeds the minimum criteria for a triage test. The 7-gene signature had satisfactory sensitivity for HIV-TB co-infection, with sensitivities of 100%, 75%, and 85% on the train, test, and validation sets respectively (FIG. 2C). However, HIV positive samples were still misclassified at a higher rate than HIV negative samples. Removing HIV+ samples from our datasets resulted in improved classification with sensitivities of 93% and 97% and specificities of 94% and 77% on the train and test sets respectively. Removal of HIV+ subjects allows the 7-gene signature to reach the WHO optimal sensitivity for a triage test and land just 3% below the optimal specificity threshold.

This model was then tested on a validation cohort and found that the 7-gene signature performed well, with an ROC-AUC of 0.893 [95% CI: 0.805-0.980], 81.7% accuracy, 89.3% sensitivity, and 72.0% specificity. The TB score was found to be highly correlated with the Xpert Ultra Semiquantitative result in the training, test, and validation datasets, which bins samples based on the cycle threshold (CT) of the first positive probe that detects M. tuberculosis as follows: “low”=22<CT≤38, “medium”=16<CT≤22, and “high”=CT≤16 (FIG. 2E). These results indicate that the 7-gene signature has the potential to serve as a triage tool for TB.

Finally, the 7-gene signature results were compared to chest X-ray (CXR) scores, as radiographs show evidence of the host response to TB. Recent developments in computer-aided detection (CAD) software have improved the performance of CXRs, but still fall short of the WHO TPPs for a triage test. Three commercially available CAD applications were used to analyze CXRs from individuals enrolled in the R2D2 and TB2 studies: CAD4 TB (Delft Imaging, Netherlands), qXR (Quire.ai, India), and Lunit Insight chest X-ray TB algorithm (Lunit Inc., South Korea). The TB abnormality score is reported on a scale of 0-100 (for CAD4 TB and Lunit) or 0-1 (for qXR), where a higher value indicates a more abnormal radiograph. Despite performance differences between each of the CAD algorithms, the 7-gene TB score is moderately correlated with the reported TB abnormality scores (Pearson r=0.54-0.68; FIG. 2F).

### Example 5: Comparison of Plasma cfRNA Biomarkers to Whole Blood RNA and Protein Studies

While many potential wbRNA signatures have been identified for the diagnosis of TB disease in clinically relevant populations, only a handful meet the minimal WHO criteria for a triage test. Unsurprisingly, the performance of these wbRNA signatures varies widely when applied to multi-country cohorts, as diagnostic performance heterogeneity between populations is a well-known barrier to the development of triage and diagnostic assays.

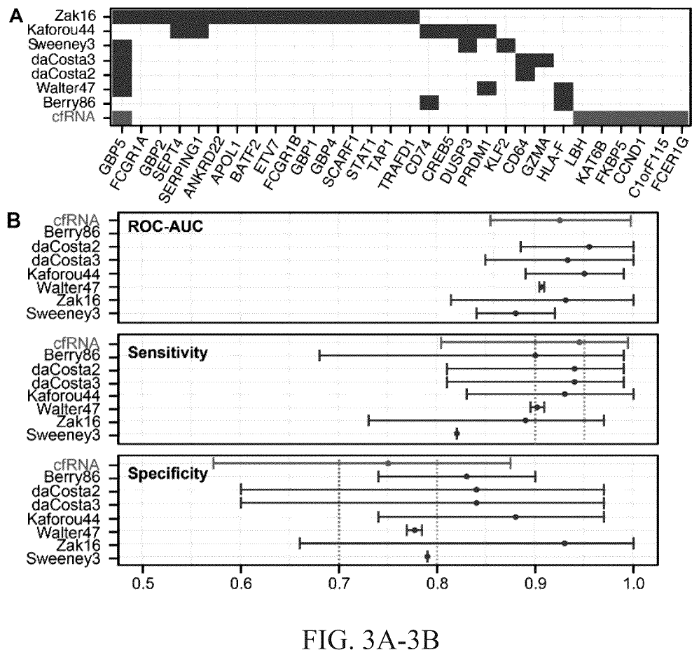

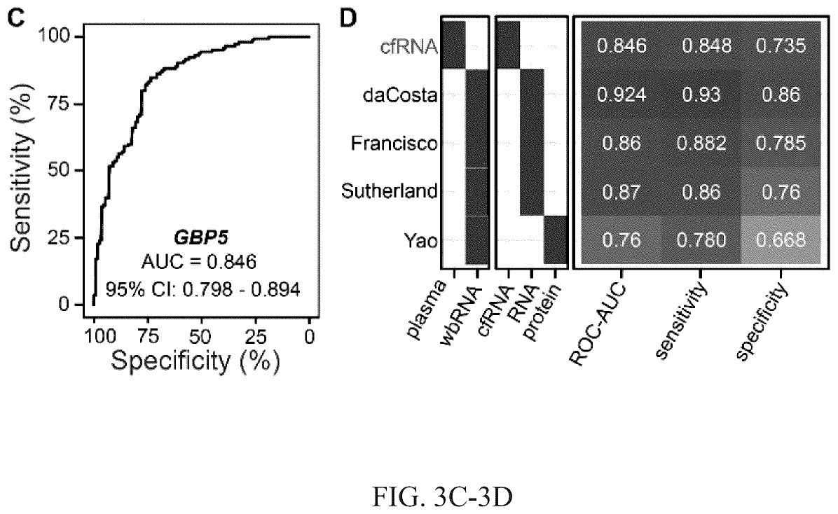

2 of the 7 genes in the signature (GBP5 and LBH) were found to overlap with those reported in previous wbRNA studies (FIG. 3A). Among the wbRNA signatures that meet the minimal WHO criteria for a triage test, 7 have been evaluated for their performance in distinguishing TB from other diseases in a multi-cohort population (FIG. 3B). The performance of these 7 signatures varies widely, with sensitivities and specificities ranging from 79.3-100% and 80-95%, respectively. Collectively, the 7 wbRNA signature gene sets and the 7-gene signature gene set include 176 genes, of which 169 are unique to a single signature set.

GBP5 was the most frequently occurring transcript, occurring in the 7-gene signature and in 5/7 whole blood signatures. GBP5 has been shown to be critical for NLRP3 inflammasome activation, which mediates caspase-1 activation and secretion of proinflammatory cytokines in response to pathogenic bacteria and cellular damage. The discriminatory power of GBP5 alone has been investigated in three of the wbRNA studies: da Costa et al. report a sensitivity of 93%, specificity of 86%, and ROC-AUC of 0.924; Francisco et al. report a sensitivity of 88.2%, specificity of 78.5%, and ROC-AUC of 0.86; and Sutherland et al. (Xpert-MTB-HR prototype using the Sweeney3 signature) reports a sensitivity of 86%, specificity of 76%, and an ROC-AUC of 0.87. In comparison, the abundance of GBP5 in plasma resulted in a sensitivity of 84.8%, specificity of 73.5%, and an ROC-AUC of 0.846 (FIGS. 3C and 3D). Whole blood GBP5 protein levels have also been evaluated as a potential biomarker for TB. Yao et al. demonstrate that whole blood GBP5 protein levels are significantly higher in people with microbiologically confirmed TB. However, the performance of the plasma protein biomarker at the optimal threshold (sensitivity=0.7802, specificity=0.6691, ROC-AUC=0.76) is poorer than the plasma RNA biomarker. This suggests that plasma cfRNA is a more effective biomarker for TB than whole blood GBP5 protein levels.

### Example 6: Evaluating the 7-Gene Signature in Plasma cfRNA and Whole Blood RNA Samples

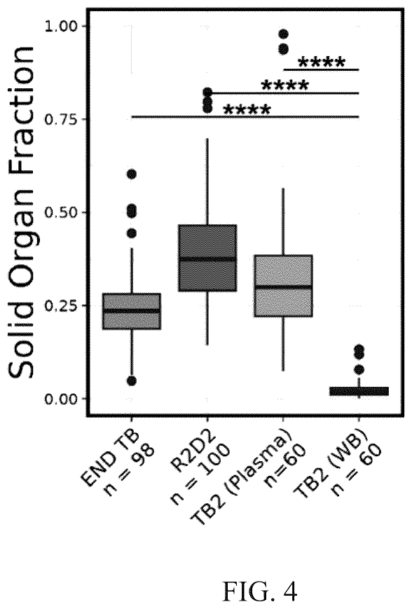

Given the limited overlap of the 7-gene signature with genes previously reported in wbRNA signatures, we evaluated differences in origin of cfRNA and wbRNA. wbRNA-sequencing was performed for 60 samples, 35 (58.33%) of which were from individuals diagnosed with microbiologically confirmed TB and 50 (83.33%) were from HIV-negative individuals. A mean of 13,507,257 reads per sample was obtained. Plasma cfRNA was found to be derived from both cells in the blood compartment and from vascularized solid tissues, while wbRNA is primarily released by circulating immune cells (FIG. 4).

### Example 7: Identification of a Broader Set of Genes by Re-Splitting the Discovery Data Using Variant Seeds

To further explore potential biomarkers for diagnosing TB, the 15 machine learning models were retrained 800 times using 400 different seeds to randomly split the discovery data set. Half of the 800 runs were conducted with a base-mean filter >100, while the other half were performed with a less-stringent filter of base-mean >50. Across 800 iterations, the GFS and GLMNET-Lasso models had the best performance and selected the smallest number of features. The 50 most frequently occurring genes as identified by the GLMNET-Lasso or the GFS model on each set of filtering conditions were selected to further explore potential biomarkers for TB. Total frequency of these genes was counted across the 800 iterations (1,600 total models). Table 2 shows the frequency of biomarker identification across 1,600 independently trained models where gene names are displayed in the “Gene” column. The “TF” column displays the total frequency, or total number of times a gene was used by either GFS or GLMNET-Lasso across the 1,600 models that were trained. There was significant overlap in genes both between model type and filter condition. A total of 107 unique biomarkers for TB were identified from this analysis with GBP5 appearing as the most frequently selected gene (Table 2).

TABLE 2

| Gene | TF | Gene | TF | Gene | TF | Gene | TF | Gene | TF |
|---|---|---|---|---|---|---|---|---|---|
| GBP5 | 1581 | DUSP3 | 130 | GBP2 | 65 | CFLAR | 41 | MLLT6 | 20 |
| CCND1 | 682 | SH3BGRL3 | 120 | ZNF451 | 64 | NCOA4 | 38 | WARS1 | 18 |
| CST3 | 419 | DYSF | 116 | S100A8 | 62 | HELZ | 37 | GBP4 | 17 |
| HEMGN | 410 | FCGR3B | 116 | STAT1 | 61 | SYNE2 | 37 | BCL11B | 15 |
| EIF1 | 320 | SNX3 | 111 | FAM13B | 60 | GAS5 | 36 | ARPC1B | 14 |
| H19 | 317 | ADAM10 | 108 | PCBP1 | 59 | BANK1 | 35 | PLAC8 | 14 |
| TMCC2 | 244 | PGD | 108 | NEAT1 | 57 | MAGI2-AS3 | 35 | VPS13C | 14 |
| ALB | 234 | UBE2J1 | 91 | GCA | 54 | ERG | 34 | FECH | 13 |
| LPP | 222 | USP12 | 85 | CPEB4 | 52 | GBP1 | 34 | GABARAP | 13 |
| FXBP5 | 205 | ATM | 84 | GREM1 | 51 | SAMD14 | 33 | SLC2A1 | 13 |
| GPI | 203 | MNDA | 83 | LBH | 51 | RPA1 | 32 | SNORD3A | 13 |
| GPX1 | 195 | NDUFAS | 82 | TPI1 | 50 | GOSR1 | 31 | IGF2 | 12 |
| KAT6B | 194 | CAPRIN1 | 80 | FCHO2 | 50 | AKT3 | 30 | KLF6 | 12 |
| RNU5A-1 | 190 | ATF4 | 78 | OPA1 | 49 | ZYX | 30 | SRSF9 | 12 |
| MEA1 | 178 | DCUN1D1 | 78 | MTND2P28 | 47 | CHMP4B | 27 | YTHDC1 | 12 |
| ABLIM1 | 175 | LYZ | 76 | DTX3L | 46 | APOL6 | 26 | FCER1G | 11 |
| LTA4H | 162 | AP1B1 | 72 | ZFHX3 | 46 | CASP4 | 25 | LMNB1 | 10 |
| SERPINB6 | 153 | WWP1 | 72 | YOD1 | 45 | NCF2 | 25 | PLAAT4 | 10 |
| JADE1 | 151 | PEA15 | 71 | ALDOA | 43 | HMGN2 | 22 | ENAH | 9 |
| NFIA | 144 | SERF2 | 69 | CAVIN1 | 42 | S100A9 | 22 | | |
| USP4 | 140 | PURA | 67 | LYPLAL1 | 42 | BNIP3L | 21 | | |
| ZC3H6 | 138 | IFITM3 | 66 | ATP6V1F | 41 | C1orf115 | 20 | | |

TF = Total Frequency

What is claimed is:

1. A method comprising:
a) obtaining cell-free RNA from a biological sample of a subject, wherein the biological sample is a plasma sample or a serum sample;
b) determining a profile of a panel of biomarkers in the cell-free RNA; and
c) determining a tuberculosis disease status in the subject based on the profile of the panel of biomarkers.

2. The method of claim 1 wherein the step of determining a profile of a panel of biomarkers comprises performing nucleotide sequencing of the cell-free RNA.

3. The method of claim 1 or 2, wherein the step of determining a profile of a panel of biomarkers comprises measuring the levels of RNAs corresponding to the biomarkers in the panel.

4. The method of claim 3, wherein the panel of biomarkers comprises one or more genes selected from the group consisting of: GBP5, CCND1, JADE1, ALB, NFIA, KAT6B, H19, PURA, FKBP5, FCGR3B, LPP, LTA4H, S100A8, S100A9, TMCC2, LBH, MEA1, GBP4, STAT1, OPA1, BCL11B, DUSP3, GBP1, SYNE2, DTX3L, DYSF, EIF1, GABARAP, IFITM3, RNU5A-1, SLC2A1, CAVIN1, GPI, IGF2, KLF6, SERPINB6, SRSF9, CST3, ERG, GCA, LYZ, MNDA, NDUFA5, ATP6V1F, LMNB1, PLAAT4, APOL6, ENAH, GBP2, HMGN2, HEMGN, ATM, USP12, BNIP3L, PGD, Clorf115, MLLT6, ALDOA, WARS1, ABLIM1, MTND2P28, YOD1, UBE2J1, ARPC1B, FAM13B, PLAC8, VPS13C, FECH, GPX1, SNORD3A, GREM1, USP4, YTHDC1, ZC3H6, FCER1G, SH3BGRL3, SNX3, PCBP1, NEAT1, CPEB4, FCHO2, TPI1, ATF4, CAPRIN1, CFLAR, PEA15, NCOA4, BANK1, RPA1, SERF2, AKT3, ZYX, CHMP4B, CASP4, NCF2, ADAM10, DCUN1D1, AP1B1, WWP1, ZNF451, ZFHX3, LYPLAL1, HELZ, GAS5, MAGI2-AS3, SAMD14, and GOSR1.

5. The method of claim 3, wherein the panel of biomarkers comprises one or more genes selected from macrophage markers, neutrophil markers, interferon genes, or antimicrobial genes.

6. The method of claim 5, wherein the macrophage markers comprise of one or more genes selected from the group consisting of MARCO, SOCS3, FCGR1A, MPO, and C1QB.

7. The method of claim 5 or 6, wherein the neutrophil markers comprise one or more genes selected from the group consisting of ELANE, FCGR3A, S100A8, S100A9, and ERG.

8. The method of any one of claims 5-7, wherein the interferon genes comprise of one or more genes selected from the group consisting of IFI27L1, IFIT2, IFIT3, IFITM3, and IRF1.

9. The method of any one of claims 5-8, wherein the antimicrobial genes comprise of one or more genes selected from the group consisting of AZU1, CTSG, DEFA4, STAT1, GBP1, GBP2, GBP4, GBP5, and GBP6.

10. The method of claim 3, wherein the panel of biomarkers comprises GBP5.

11. The method of claim 10, wherein the panel of biomarkers comprises GBP5 and one or more additional genes.

12. The method of claim 11, wherein the panel of biomarkers comprises GBP5 and 2-8 additional genes.

13. The method of claim 11, wherein the panel of biomarkers comprises GBP5 and one or more genes selected from the group consisting of:
i) LBH, KAT6B, FKBP5, CCND1, Clorf115, and FCER1G; or
ii) DYSF, SMARCD3, VAMP5, FCGR3B, GPI, MPO, CREB5, and GBP2.

14. The method of claim 3, wherein the panel of biomarkers comprises one or more genes selected from the group consisting of:
i) GBP5, LBH, KAT6B, FKBP5, CCND1, Clorf115, and FCER1G; or
ii) GBP5, DYSF, SMARCD3, VAMP5, FCGR3B, GPI, MPO, CREB5, and GBP2.

15. The method of any one of the previous claims, wherein determining a tuberculosis disease status in the subject based on the profile of the panel of biomarkers comprises comparing the profile of the panel of biomarkers in the cell free RNA from the biological sample to a reference profile of the panel of biomarkers.

16. The method of any one of the previous claims, wherein determining a tuberculosis disease status in the subject based on the profile of the panel of biomarkers comprises comparing the levels of RNAs corresponding to the biomarkers in the panel to levels of RNAs corresponding to the biomarkers in the reference profile.

17. The method of any one of the previous claims, wherein a tuberculosis disease status comprises a tuberculosis free status, a latent tuberculosis status, an active tuberculosis status, or a tuberculosis progressing status.

18. The method of any one of the previous claims, wherein the biological sample is a plasma sample.

19. The method of any one of the previous claims, wherein the biological sample is a serum sample.

20. The method of any one of the previous claims, wherein the subject is a mammal.

21. The method of any one of the previous claims, wherein the subject is a human.

22. The method of any one of claims 1-21, wherein the profile of the panel of biomarkers comprises subject-specific gene expression of the biomarkers in the panel.

23. The method of any one of claims 1-21, wherein the profile of the panel of biomarkers comprises a score determined based on the levels of RNAs corresponding to the biomarkers in the panel using a trained classification model, and wherein the score correlates with disease status.

24. The method of any one of the previous claims, wherein the cell-free RNA is substantially free of contaminant RNA.

25. The method of any one of the previous claims, wherein the tuberculosis disease status determined is used to triage the subject.

26. The method of any one of the previous claims, wherein the tuberculosis disease status determined is used in determining treatment for the subject.

27. The method of any one of the previous claims, wherein the tuberculosis disease status determined is used to monitor treatment of the subject.

28. The method of any one of the previous claims, wherein the tuberculosis disease status determined is used to monitor efficacy of a treatment of the subject.

29. The method of any one of the previous claims, wherein test performance of the trained classification model is measured through one or more of accuracy, sensitivity, specificity, and/or area under the receiver operating characteristic curve (ROC-AUC).

30. The method of any one of the previous claims, wherein the tuberculosis disease status is determined at a greater than 80%, 85%, 90%, 91%, 92%, 93%, 94%, or 95% accuracy.

31. The method of any one of the previous claims, wherein the tuberculosis disease status is determined at a greater than 80%, 85%, 90%, 91%, 92%, 93%, 94%, or 95% sensitivity.

32. The method of any one of the previous claims, wherein the tuberculosis disease status is determined at a greater than 70%, 75%, 80%, 85%, 90%, 91%, 92%, 93%, 94%, or 95% specificity.

33. The method of any one of the previous claims, wherein the trained classification model comprises one or more models selected from the group consisting of generalized linear models with Ridge and LASSO feature selection (GLMNETRIDGE and GLMNETLASSO), support vector machines with linear and radial basis function kernel (SVMLin and SVMRAD), random forest (RF), random forest ExtraTrees (EXTRATREES), neural networks (NNET), linear discriminant analysis (LDA), nearest shrunken centroids (PAM), C5.0 (C5), k-nearest neighbors (KNN), naive bayes (NB), CART (RPART), generalized linear model (GLM), and greedy forward search algorithm (GFS).

34. The method of any one of the previous claims, wherein the method further comprises step d) treating the subject based on the tuberculosis disease status.

35. The method of claim 34, wherein step d) treating the subject based on the tuberculosis disease comprises treating the subject with an antibiotic.

36. A method for establishing a panel of biomarkers that correlates with tuberculosis disease status, comprising
a) obtaining cell-free RNA from a biological sample of each of a group of subjects, wherein the biological sample is a plasma sample or a serum sample, and wherein the group of subjects comprise subjects positive for tuberculosis and subjects negative for tuberculosis;
b) detecting levels of cell-free RNAs of a plurality of genes in the subjects;
c) dividing the subjects into a training set, and a testing set, wherein each set comprises subjects positive for tuberculosis and subjects negative for tuberculosis;
d) based on levels of cell-free RNAs of a plurality of genes in the training set, selecting a panel of genes, wherein the selecting comprises:
selecting differentially abundant genes based on a differential analysis; and
training a classification model using the levels of cell-free RNAs of the selected genes; thereby determining a classification score for optimal classifier performance in an ROC (Receiver Operating Characteristics) analysis;
e) testing the trained classification model by applying the trained model and the classification score to the cell-free RNA levels of the testing set, and determining the panel of genes as the panel of biomarkers that correlates with tuberculosis disease status if the trained classification model provides differentiation in the testing between subjects positive for tuberculosis and subjects negative for tuberculosis with at least 85% sensitivity and at least 65% specificity.

37. The method of claim 36, wherein the differentially abundant genes are selected by removing genes of low count and genes having an adjusted p-value greater than 0.05.

38. The method of claim 36 or 37, wherein classification model comprises one or more models selected from the group consisting of generalized linear models with Ridge and LASSO feature selection (GLMNETRIDGE and GLMNETLASSO), support vector machines with linear and radial basis function kernel (SVMLin and SVMRAD), random forest (RF), random forest ExtraTrees (EXTRATREES), neural networks (NNET), linear discriminant analysis (LDA), nearest shrunken centroids (PAM), C5.0 (C5), k-nearest neighbors (KNN), naive bayes (NB), CART (RPART), generalized linear model (GLM), and greedy forward search algorithm (GFS).

39. The method of any one of claims 36-38, wherein the group of subjects include at least 100 subjects positive for TB, and at least 100 subjects negative for TB.

40. The method of any one of claims 36-39, wherein the panel of biomarkers comprises at least 1 gene.

41. The method of any one of claims 36-40, wherein the trained classification model provides differentiation in the testing between subjects positive for tuberculosis and subjects negative for tuberculosis with at least 80% sensitivity.

42. The method of any one of claims 36-40, wherein the trained classification model provides differentiation in the testing between subjects positive for tuberculosis and subjects negative for tuberculosis with at least 60% specificity.

43. The method of any one of claims 36-42, wherein the trained classification model provides differentiation in the testing between subjects positive for tuberculosis and subjects negative for tuberculosis with at least 90% sensitivity and at least 70% specificity

44. The method of any one of claims 36-42, when step c) further comprises dividing the subjects into a training set, a validation set, and a testing set, wherein each set comprises subjects positive for tuberculosis and subjects negative for tuberculosis.

45. The method of claim 44, wherein step (e) further comprises testing the trained classification model by applying the trained model and the classification score to the cell-free RNA levels of the validation set and the testing set, and determining the panel of genes as the panel of biomarkers that correlates with tuberculosis disease status if the trained classification model provides differentiation in the testing between subjects positive for tuberculosis and subjects negative for tuberculosis with at least 85% sensitivity and at least 65% specificity

46. The method of any one of claims 36-42 or 44-45, wherein the trained classification model does not provide differentiation in the testing between subjects positive for tuberculosis and subjects negative for tuberculosis with at least 85% sensitivity and at least 65% specificity, the method further comprises one or a combination of the steps:
f) selecting a different modeling technique;
g) adjusting the hyperparameters for training the model;
h) changing the initial filtering steps and how Differential abundance analysis is performed; and/or
i) adjusting the classifier score threshold based on the training set.
